(19) United States
(12) Patent Application Publication
(10) Pub. No.: US 2014/0287931 A1
(43) Pub. Date: Sep. 25, 2014

(54) METHODS AND COMPOSITIONS FOR PREDICTING RESISTANCE TO ANTICANCER TREATMENT

(71) Applicants: Rene Bernards, Amsterdam (NL); Sidong Huang, Amsterdam (NL); Michael Holzel, Amsterdam (NL); Katrien Berns, Amsterdam (NL)

(72) Inventors: Rene Bernards, Amsterdam (NL); Sidong Huang, Amsterdam (NL); Michael Holzel, Amsterdam (NL); Katrien Berns, Amsterdam (NL)

(73) Assignee: Stichting het Nederlands Kanker Instituut - Antoni van Leeuwenhoek ziekenhuis, Amsterdam (NL)

(21) Appl. No.: 14/009,465

(22) PCT Filed: Apr. 4, 2012
(86) PCT No.: PCT/US12/32212

§ 371 Date: Jun. 3, 2014

Related U.S. Application Data
(60) Provisional application No. 61/471,615, filed on Apr. 4, 2011.

(51) Int. Cl. C12Q 1/68; G01N 33/574

(52) U.S. Cl. CPC C12Q 1/6886; G01N 33/57496; G01N 33/57423; G01N 33/57415
USPC 506/2; 435/6.11; 506/9; 435/6.12; 435/7.1; 435/18; 435/7.92

(57) ABSTRACT

The instant application provides methods and related compositions pertaining to the identification of resistance to anticancer treatment in. a patient. In a particular embodiment, the invention provides biomarkers for the identification of resistance to anticancer treatment in a breast cancer patient, wherein a reduced expression of a MEDIATOR and/or SWI/SNF complex gene in the breast cancer cells of the patient indicates that the breast cancer cells in the patient may be successfully treated with a PI3K and/or mTOR inhibitor.

### CROSS-REFERENCE TO RELATED APPLICATION

This application claims the benefit of priority of U.S. Provisional Application Ser. No. 61/471,615 filed Apr. 4, 2011, which is incorporated herein by reference in its entirety.

### FIELD OF THE INVENTION

The invention relates to the field of methods and related compositions for predicting resistance to anticancer treatment. In certain embodiments, the invention relates to the field of methods and related compositions for predicting resistance to anticancer treatment in a cancer patient by detecting a reduced expression level of a SWI/SNF complex and/or MEDIATOR complex and/or RAS-GAP gene and/or protein in one or more cancer cells of the patient. In other embodiments, the invention relates to the field of methods and related compositions for predicting resistance to anticancer treatment by detecting one or more inactivating mutations in a SWI/SNF complex and/or MEDIATOR complex and/or RAS-GAP gene. In some embodiments, the invention relates to the field of methods and related compositions for predicting resistance to anticancer treatment by detecting dysfunction and/or inactivity of one or more SWI/SNF complex and/or MEDIATOR complex and/or RAS-GAP proteins.

### BACKGROUND OF THE INVENTION

Activation of signaling pathways in cancer is often the result of genomic alterations (mutations, translocations, copy number gains and/or losses) in key components of these pathways. Cancer cells often depend on the continued presence of the signals that emanate from these genomic alterations and sudden inhibition frequently results in death of the cancer cells, a phenomenon coined “oncogene addiction” (Sharma and Settleman, 2007). The presence of specific changes in the genomes of cancer cells can therefore have strong predictive value for responsiveness to therapies that target these mutations (Pao and Chmielecki).

Such drug response biomarkers are urgently needed for the rational selection of patients for these therapies, as their clinical benefit is often limited due to the fact that only a subset of patients responds. Considering the high cost of targeted therapeutics, response biomarkers are not only a clinical necessity, but also an economic requirement to keep the cost of cancer care in check by reducing the number of patients that receive expensive drugs without experiencing therapeutic benefit.

Lung cancer is a leading cause of cancer deaths worldwide and tobacco smoking remains the major risk factor. Genomic alterations of receptor tyrosine kinases are frequently found in non-small cell lung cancers, the predominant histological subtype, and are particularly enriched (˜40%) in non-smokers (Rudin et al., 2009). Lung cancers with activating mutations of the EGFR (epidermal growth factor receptor) respond well to treatment with EGFR inhibitors (gefitinib and erlotinib) in the clinic and constitute the largest subgroup of patients (˜10%-20%) tractable for an effective tyrosine kinase inhibitor therapy (Lynch et al., 2004; Maemondo et al.; Resell et al., 2009; Sharma et al., 2007). Recently, EML4-ALK translocations were identified in ˜2%-5% of NSCLC providing a second promising molecular target for the treatment of NSCLC (Soda et al., 2007). The fusion of the N-terminal part of EML4 (echinoderm microtubule associated protein like 4) with the C-terminal kinase domain of ALK (anaplastic large cell lymphoma kinase) results in the stable dimerization and constitutive activation of the EML4-ALK fusion kinase. The dual tyrosine kinase inhibitor crizotinib potently inhibits ALK/MET and is currently evaluated in clinical trials. The first phase I study with crizotinib in EML4-ALK positive advanced NSCLC demonstrated remarkable activity (Kwak et al.).

Despite these encouraging clinical results, lung cancers with EGFR mutations or EML4-ALK translocations do not respond equally well to these inhibitors (primary resistance) and all tumors develop resistance (acquired resistance) under prolonged treatment (Jackman et al.). Several acquired resistance mechanisms were identified in pre-clinical studies and also confirmed in specimens from relapsed patients that initially responded well to EGFR or ALK inhibitor treatment. Second site mutations of the EGFR (EGFR^{T790M}) and MET amplifications account for ˜50% of the cases of acquired resistance to EGFR inhibitors (Engelman et al., 2007; Hammerman et al., 2009; Kobayashi et al., 2005). The EGFR^{T790M} gatekeeper mutation prevents binding of the inhibitors to the kinase domain, but preserves the activity of the kinase. The frequency of EML4-ALK second site mutations in relapsed tumors is currently unknown and was only found in a single case so far (Choi et al.).

Nevertheless, in a large number of cases the mechanism of resistance to EGFR or ALK inhibitors remains unknown and in particular the determinants of primary resistance are obscure. Understanding the relevant genes and signaling pathways that contribute to resistance of NSCLC cells to tyrosine kinase inhibitors might not only provide drug response markers to stratify treatment options, but might also delineate new therapeutic strategies to overcome the drug resistance.

Citation or identification of any document in this application is not an admission that such document is available as prior art to the present invention.

### SUMMARY OF THE INVENTION

In certain embodiments, the invention provides a method of evaluating and/or predicting resistance to an Akt activation and/or mTOR inhibitor in a patient in need thereof, comprising (a) measuring expression levels of one or more SWI/SNF complex and/or MEDIATOR complex nucleic acid and/or proteins in the patient; and (b) comparing the expression levels of the one or more SWI/SNF complex and/or MEDIATOR complex nucleic acid and/or proteins in (a) with the expression levels of one or more reference SWI/SNF complex and/or MEDIATOR complex nucleic acid and/or proteins, wherein the one or more reference SWI/SNF complex and/or MEDIATOR complex nucleic acid and/or proteins are from a control sample, wherein a reduction in the expression of the one or more SWI/SNF complex and/or MEDIATOR complex nucleic acid and/or proteins in comparison to the one or more reference SWI/SNF complex and/or MEDIATOR complex nucleic acid and/or proteins is indicative of resistance to an Akt activation and/or mTOR inhibitor in the patient.

In other embodiments, the invention provides a method of evaluating and/or predicting resistance to an Akt activation and/or mTOR inhibitor in a patient in need thereof, comprising (a) isolating nucleic acid from the patient, wherein the nucleic acid comprises one or more SWI/SNF complex and/or MEDIATOR complex DNA and/or RNA; and (b) analyzing the nucleic acid of (a) for the presence of one or more inactivating mutations in the SWI/SNF complex and/or MEDIATOR complex DNA and/or RNA, wherein the presence of one or more inactivating mutations in the one or more SWI/SNF complex and/or MEDIATOR complex DNA and/or RNA analyzed in (b) is indicative of resistance to an Akt activation and/or mTOR inhibitor in the patient.

In some embodiments, the invention provides a method of evaluating and/or predicting resistance to an Akt activation and/or mTOR inhibitor in a patient in need thereof, comprising (a) isolating protein from the patient, wherein the protein comprises one or more SWI/SNF complex and/or MEDIATOR complex proteins; (b) analyzing the activity of the one or more SWI/SNF complex and/or MEDIATOR complex proteins in (a); and (c) comparing the activity of the one or more SWI/SNF complex and/or MEDIATOR complex proteins in (b) with the activity of one or more reference SWI/SNF complex and/or MEDIATOR complex proteins, wherein a difference in activity of the one or more SWI/SNF complex and/or MEDIATOR complex proteins from (b) in comparison to the one or more SWI/SNF complex and/or MEDIATOR complex reference proteins in (c) is indicative of resistance to an Akt activation and/or mTOR inhibitor in the patient.

In certain embodiments, the expression levels of one or more SWI/SNF complex nucleic acids (e.g., DNA, RNA) and/or proteins are measured.

In certain embodiments, the expression levels of one or more MEDIATOR complex nucleic acids (e.g., DNA, RNA) and/or proteins are measured.

In some embodiments, the invention provides a method of evaluating and/or predicting resistance to an Akt activation and/or mTOR inhibitor in a patient in need thereof, comprising (a) measuring expression levels of one or more RAS-GAP nucleic acid and/or proteins in the patient; and (b) comparing the expression levels of the one or more RAS-GAP nucleic acid and/or proteins in (a) with the expression levels of one or more reference RAS-GAP nucleic acid and/or proteins, wherein the one or more reference RAS-GAP nucleic acid and/or proteins are from a control sample, wherein a reduction in the expression of the one or more RAS-GAP nucleic acid and/or proteins in comparison to the one or more reference RAS-GAP nucleic acid and/or proteins is indicative of resistance to an Akt activation and/or mTOR inhibitor in the patient.

In other embodiments, the invention provides a method of evaluating and/or predicting resistance to an Akt activation and/or mTOR inhibitor in a patient in need thereof, comprising (a) isolating nucleic acid from the patient, wherein the nucleic acid comprises one or more RAS-GAP DNA and/or RNA; and (b) analyzing the nucleic acid of (a) for the presence of one or more inactivating mutations in the RAS-GAP DNA and/or RNA, wherein the presence of one or more inactivating mutations in the one or more RAS-GAP DNA and/or RNA analyzed in (b) is indicative of resistance to an Akt activation and/or mTOR inhibitor in the patient.

In yet other embodiments, the invention provides a method of evaluating and/or predicting resistance to an Akt activation and/or mTOR inhibitor in a patient in need thereof, comprising (a) isolating protein from the patient, wherein the protein comprises one or more RAS-GAP proteins; (b) analyzing the activity of the one or more RAS-GAP proteins in (a); and (c) comparing the activity of the one or more RAS-GAP proteins in (b) with the activity of one or more reference RAS-GAP proteins, wherein a difference in activity of the one or more RAS-GAP proteins from (b) in comparison to the one or more RAS-GAP reference proteins in (c) is indicative of resistance to an Akt activation and/or mTOR inhibitor in the patient.

In some embodiments, the expression levels of one or more RAS-GAP nucleic acids (e.g., DNA, RNA) are measured. In other embodiments, the expression levels of one or more RAS-GAP proteins are measured.

In some embodiments of the methods described herein for evaluating and/or predicting resistance to anticancer treatment in a patient in need thereof, the patient has lung cancer (e.g., non-small-cell lung cancer), breast cancer, ovarian cancer, bladder cancer, colorectal cancer, cervical cancer, mesothelioma, solid tumors, liver cancer, renal cell carcinoma, stomach cancer, head and neck cancer, sarcoma, prostate cancer, subependymal giant cell astrocytoma, endometrial cancer, melanoma, thyroid cancer, brain cancer, adenocarcinoma, glioma, glioblastoma, esophageal cancer, neuroblastoma, lymphoma, and/or a hematological cancer.

In some embodiments, the resistance to an Akt activation and/or mTOR inhibitor is resistance to treatment with a receptor tyrosine kinase inhibitor. Examples of receptor tyrosine kinase inhibitors include gefitinib, erlotinib, EKB-569, lapatinib, CI-1033, cetuximab, panitumumab, PKI-166, AEE788, sunitinib, sorafenib, dasatinib, nilotinib, pazopanib, vandetaniv, cediranib, afatinib, motesanib, CUDC-101, imatinib mesylate, crizotinib, ASP-3026, LDK378, AF802, and CEP37440.

In some embodiments, the inhibitor of Akt activation is a PI3K inhibitor. Examples of PI3K inhibitors include NVP-BKM120, XL147 (SAR245408), PX-866, GDC-0941, CAL-101, CNX-1351, ETP-46992, RP-5002, XL-499, and ONC-201. BEZ235, BGT226, SF1126, GSK1059615, PKI-402, PX866, GDC0941/GDC080, BKM120, NVP-BEZ235, NVP-BGT226, PF-04691502, PF-04979064, PF-05177624, PF-05197281, PF-05212384, XL 147, XL765, EXEL-1229, EXEL-2400, EXEL-3751, EXEL-4251, PWT-33597, and SB2343.

Examples of inhibitors of mTOR include rapamycin/sirolimus, temsirolimus, everolimus, PP242, PP30, INK128, WYE-600, WYE-687, WYE-354, INK128, AZD8055, Torin-1, AZD2014, ridaforolimus, OSI-027, NV-128, NV-344, AP-23675, AP-23841, AP-24170, and TAFA-93. BEZ235, BGT226, SF1126, GSK1059615, PKI-402, PX866, GDC0941/GDC080, BKM120, NVP-BEZ235, NVP-BGT226, PF-04691502, PF-04979064, PF-05177624, PF-05197281, PF-05212384, XL147, XL765, EXEL-1229, EXEL-2400, EXEL-3751, EXEL-4251, PWT-33597, and SB2343.

In some embodiments, the resistance to an Akt activation and/or mTOR inhibitor is resistance to treatment with an inhibitor of Akt activation. In certain embodiments, the inhibitor of Akt activation inhibits a cellular protein that interacts directly with Akt. In other embodiments, the inhibitor of Akt activation inhibits a cellular protein that interacts indirectly with Akt. In yet other embodiments, the inhibitor of Akt activation is a receptor tyrosine kinase inhibitor.

Examples of SWI/SNF complex nucleic acids and/or proteins include ARID1A, ARID1B, ARID2, SMARCA2, SMARCA4, PBRM1, SMARCC2, SMARCC1, SMARCD1, SMARCD2, SMARCD3, SMARCE1, ACTL6A, ACTL6B, and SMARCB1.

Examples of MEDIATOR complex nucleic acids and/or proteins include MED22, MED11, MED17, MED20, MED30, MED19, MED18, MED8, MED6, MED28, MED9, MED21, MED4, MED7, MED31, MED10, MED1, MED26, MED2, MED3, MED25, MED23, MED5, MED14, MED16, MED15, CycC, CDKS, MED13, MED12, MED12L, and MED13L.

Examples of RAS-GAP nucleic acids and/or proteins include DAB2IP, NF1, and RASAL3.

In some embodiments, analyzing nucleic acid comprises sequencing the nucleic acid. In other embodiments, analyzing nucleic acid comprises subjecting the nucleic acid to MLPA. In yet other embodiments, analyzing nucleic acid comprises subjecting the nucleic acid to CGH. In certain embodiments, analyzing nucleic acid comprises subjecting the nucleic acid to FISH.

In certain embodiments, an inactivating mutation is selected from the group consisting of: point mutations, translocations, amplifications, deletions, and hypomorphic mutations.

In certain embodiments, nucleic acid in a method of the invention comprises one or more SWI/SNF complex genes. In other embodiments, the nucleic acid comprises one or more MEDIATOR complex genes. In yet other embodiments, the nucleic acid comprises one or more RAS-GAP genes. In certain embodiments, one or more SWI/SNF complex and/or MEDIATOR complex proteins analyzed are inactive. In further embodiments, the one or more SWI/SNF complex and/or MEDIATOR complex proteins are inactive due to one or more posttranslational modifications. In some embodiments, one or more RAS-GAP proteins analyzed are inactive. In further embodiments, the one or more RAS-GAP proteins are inactive due to one or more posttranslational modifications. In some embodiments, the invention relates to a microarray comprising a plurality of polynucleotide probes each complementary and hybridizable to a sequence in a different gene that is a SWI/SNF complex gene that is a marker for resistance to an Akt activation and/or mTOR inhibitor in a patient that has cancer.

In other embodiments, the invention relates to a microarray comprising a plurality of polynucleotide probes each complementary and hybridizable to a sequence in a different gene that is a MEDIATOR complex gene that is a marker for resistance to an Akt activation and/or mTOR inhibitor in a patient that has cancer.

In some embodiments, the invention relates to a microarray comprising a plurality of polynucleotide probes each complementary and hybridizable to a sequence in a different gene that is a SWI/SNF complex and/or MEDIATOR complex gene that is a marker for resistance to resistance to an Akt activation and/or mTOR inhibitor in a patient that has cancer.

In other embodiments, the invention relates to a microarray comprising a plurality of polynucleotide probes each complementary and hybridizable to a sequence in a different gene that is a RAS-GAP gene that is a marker for resistance to resistance to an Akt activation and/or mTOR inhibitor in a patient that has cancer.

In certain embodiments, a microarray of the invention comprises a plurality of probes, wherein the plurality of probes is at least 70%, at least 80%, at least 90%, at least 95%, or at least 98% of the probes on the microarray.

In certain embodiments, in a microarray of the invention, the SWI/SNF complex gene that is a marker for resistance to anticancer treatment is selected from the group consisting of ARID1A, ARID1B, ARID2, SMARCA2, SMARCA4, PBRM1, SMARCC2, SMARCC1, SMARCD1, SMARCD2, SMARCD3, SMARCE1, ACTL6A, ACTL6B, and SMARCB1.

In other embodiments, in a microarray of the invention, the MEDIATOR complex gene that is a marker for resistance to anticancer treatment is selected from the group consisting of MED22, MED11, MED17, MED20, MED30, MED19, MED18, MED8, MED6, MED28, MED9, MED21, MED4, MED7, MED31, MED10, MED, MED26, MED2, MED3, MED25, MED23, MED5, MED14, MED16, MED15, CycC, CDK8, MED13, MED12, MED13L, and MED12L.

In still other embodiments, in a microarray of the invention, the RAS-GAP gene is selected from the group consisting of: DAB2IP, NF1, and RASAL3.

In some embodiments, the invention relates to a kit, comprising at least one pair of primers specific for a SWI/SNF complex gene that is a marker for resistance to resistance to an Akt activation and/or mTOR inhibitor in a patient that has cancer, at least one reagent for amplification of the SWI/SNF complex gene, and instructions for use.

In other embodiments, the invention relates to a kit, comprising at least one pair of primers specific for a MEDIATOR complex gene that is a marker for resistance to resistance to an Akt activation and/or mTOR inhibitor in a patient that has cancer, at least one reagent for amplification of the MEDIATOR complex gene, and instructions for use.

In some embodiments, the invention relates to a kit, comprising at least one pair of primers specific for a SWI/SNF complex and/or a MEDIATOR complex gene that is a marker for resistance to an Akt activation and/or mTOR inhibitor in a patient that has cancer, at least one reagent for amplification of the SWI/SNF complex and/or MEDIATOR complex gene, and instructions for use.

In other embodiments, the invention relates to a kit, comprising at least one pair of primers specific for a RAS-GAP gene that is a marker for resistance to an Akt activation and/or mTOR inhibitor in a patient that has cancer, at least one reagent for amplification of the RAS-GAP gene, and instructions for use.

In certain embodiments, in a kit of the invention, the primers are specific for a SWL/SNF complex gene selected from the group consisting of ARID1A, ARID1B, ARID2, SMARCA2, SMARCA4, PBRM1, SMARCC2, SMARCC1, SMARCD1, SMARCD2, SMARCD3, SMARCE1, ACTL6A, ACTL6B, and SMARCB1.

In certain embodiments, in a kit of the invention, the primers are specific for a MEDIATOR complex gene selected from the group consisting of MED22, MED11, MED17, MED20, MED30, MED19, MED18, MED8, MED6, MED28, MED9, MED21, MED4, MED7, MED31, MED10, MED1, MED26, MED2, MED3, MED25, MED23, MED5, MED14, MED16, MEDIS, CycC, CDK8, MED13, MED12, MED13L, and MED12L.

In certain embodiments, in a kit of the invention, the primers are specific for a RAS-GAP gene selected from the group consisting of: DAB2IP, NF1, and RASAL3.

In certain embodiments, in a kit of the invention, the marker for resistance to an Akt activation and/or mTOR inhibitor is a marker for resistance to a receptor tyrosine kinase inhibitor.

In certain embodiments, in a kit of the invention, the marker for resistance to an Akt activation inhibitor is a marker for resistance to a PI3K inhibitor.

In certain embodiments, in a kit of the invention, the marker for resistance to an Akt activation and/or mTOR inhibitor is a marker for resistance to an Akt activation inhibitor. In some embodiments, the inhibitor of Akt activation inhibits a cellular protein that interacts directly with Akt. In some embodiments, the inhibitor of Akt activation inhibits a cellular protein that interacts indirectly with Akt. In other embodiments, the inhibitor of Akt activation is a receptor tyrosine kinase inhibitor.

In certain embodiments, the kit is a PCR kit. In other embodiments, the kit is an MLPA kit. In yet other embodiments, the kit is an RT-MLPA kit.

In some embodiments, the level of expression of one or more SWI/SNF complex and/or MEDIATOR complex and/or RAS-GAP genes is measured by determination of their level of transcription, using a DNA array. In other embodiments, the level of expression of one or more SWI/SNF complex and/or MEDIATOR complex and/or RAS-GAP genes is measured by determination of their level of transcription, using quantitative RT-PCR.

In some embodiments the level of expression of one or more SWI/SNF complex and/or MEDIATOR complex and/or RAS-GAP genes in a method of the invention is measured in a tumor sample from the patient. In certain further embodiments, the tumor sample is a breast tumor sample.

In certain embodiments, in the methods of the invention, the expression levels of SWI/SNF and/or MEDIATOR complex or RAS-GAP nucleic acid and/or proteins are measured in one or more cancer cells of the patient. In some embodiments, nucleic acid is isolated from one or more cancer cells of the patient. In other embodiments, protein is isolated from one or more cancer cells of the patient.

In certain embodiments, in a method of the invention, resistance to anticancer treatment in one or more cancer cells in a patient is primary resistance to anticancer treatment. In other embodiments, the resistance is secondary resistance to anticancer treatment.

These and other embodiments are disclosed or are obvious from and encompassed by, the following Detailed Description.

### BRIEF DESCRIPTION OF THE DRAWINGS

FIG. 1 depicts the results of a genome-wide RNAi screen that identifies MED12, ARID1A and SMARCE1 as critical determinants of drug sensitivity to ALK inhibitors in EML4-ALK mutant NSCLC cells. (A) Schematic outline of the ALK inhibitor resistance barcode screen performed in H3122 cells. Human shRNA library polyclonal virus was produced to infect H3122 cells, which were then left untreated (control) or treated with 5 nM NVP-TAE684. After 4 weeks of selection, shRNA inserts from both populations were recovered, labeled and hybridized to DNA. (B) Analysis of the relative abundance of the recovered shRNA cassettes from ALK inhibitor barcode experiment. Averaged data from three independent experiments were normalized and 2 log transformed. Among the 49 top shRNA candidates (M>1.5 and A>7), two independent shMED12, one shARID1A and one shSMARCE) vectors were identified. (C) Individual shRNAs from the library targeting MED12, ARID1A and SMARCE1 confer resistance to ALK inhibitors. H3122 cells expressing the empty vector pRS, control shGFP, shMED12#1, shMED12#2, shARID1A or shSMARCE1, were left untreated for 2 weeks or treated with 300 nM Crizotinib or 2.5 nM NVP-TAE684 for 4 weeks, after which the cells were fixed, stained and photographed.

FIG. 2 depicts that suppression of MED12 confers drug resistance to ALK inhibitors in EML4-ALK mutant NSCLC cells. (A) Validation of independent retroviral shRNAs (in pRS vector) targeting MED12 in H3122 cells. The functional phenotypes of non-overlapping shMED12 vectors are indicated by the colony formation assay in 300 nM Crizotinib or 2.5 nM NVP-TAE684. The cells were fixed, stained and photographed after 2 weeks (untreated) or 4 weeks (ALK inhibitors treatment). (B and C) The knockdown ability of each of the shRNAs was measured by examining the MED12 mRNA levels by qRT-PCR (B) and the MED12 protein levels by western blotting (C). Error bars denote standard deviation (SD). (D) Validation of independent lentiviral shRNAs (in pLKO vector) targeting MED12. The functional phenotypes of non-overlapping shMED12 vectors are indicated by the colony formation assay in 300 nM Crizotinib or 2.5 nM NVP-TAE684. The cells were fixed, stained and photographed after 2 weeks (untreated) or 4 weeks (ALK inhibitors treatment). (E and F) The knockdown ability of each of the shRNAs was measured by examining the MED12 mRNA levels by qRT-PCR (B) and the MED12 protein levels by western blotting. Error bars denote standard deviation (SD).

FIG. 3 shows that restoration of Med12 reverses the resistance to ALK inhibitors driven by MED12 knockdown in EML4-ALK mutant NSCLC cells. (A) Ectopic expression of mouse Med12 re-sensitizes the MED12 knockdown cells to ALK inhibitors. H3122 cells expressing pLKO control or shMED12 vectors were retrovirally infected with viruses containing pMX or pMX-Med12, and were grown in the absence or presence of 300 nM Crizotinib or 2.5 nM NVP-TAE684. Cells were then fixed, stained and photographed after 2 weeks (untreated) or 4 weeks (ALK inhibitors treatment). (B) The MED12/Med12 protein levels in H3122 cells (untreated) described in FIG. 3A. (C and D) The endogenous MED12 mRNA (C) and the exogenous Med12 mRNA were measured by qRT-PCR.

FIG. 4 shows that suppression of ARID1A or SMARCE1 confers drug resistance to ALK inhibitors in EML4-ALK mutant NSCLC cells. (A) Validation of independent retroviral shRNAs targeting ARID1A or SMARCE1 in H3122 cells. The functional phenotypes of non-overlapping shARID1A and shSMARCE1 vectors are indicated by the colony formation assay in 300 nM Crizotinib or 2.5 nM NVP-TAE684. The cells were fixed, stained and photographed after 2 weeks (untreated) or 4 weeks (ALK inhibitors treatment). (B and C) The knockdown ability of each of the shRNAs was measured by examining the ARID1A mRNA levels by qRT-PCR (B) and the ARIDLA protein levels by western blotting (C). Error bars denote standard deviation (SD). (D and E) The knockdown ability of each of the shRNAs was measured by examining the SMARCE1 mRNA levels by qRT-PCR (D) and the SMARCE1 protein levels by western blotting (E). Error bars denote standard deviation (SD).

FIG. 5 shows that restoration of SMARCE1 reverses the resistance to ALK inhibitors driven by SMACRE1 knockdown in EML4-ALK mutant NSCLC cells. (A) Ectopic expression of SMARCE1-ND that cannot be targeted by shSM4RCE1 vectors re-sensitizes the SMARCE knockdown cells to ALK inhibitors. H3122 cells expressing pRS control or shSMARCE1 vectors were retrovirally infected with viruses containing pMX or pMX-SMARCE1-ND, and were grown in the absence or presence of 300 nM Crizotinib or 2.5 nM NVP-TAE684. Cells were then fixed, stained and photographed after 2 weeks (untreated) or 4 weeks (ALK inhibitors treatment). (B) The SMARCE1 protein levels in H3122 cells (untreated) described in FIG. 3A. (C and D) The endogenous SMARCE1 mRNA was measured by qRT-PCR using a 3′ UTR specific primer set (C) and the total SMARCE1 mRNA was measured by qRT-PCR using an ORF specific primer set.

FIG. 6 shows that restoration of Med12 reverses the resistance to EGFR inhibitor driven by MED12 knockdown in PC9 EGFR mutant cells. (A) Ectopic expression of mouse Med12 re-sensitizes the otherwise resistant MED12 knockdown cells to EGFR inhibitors. PC9 cells expressing pLKO control or shMED12 vectors were retrovirally infected with viruses containing pMX or pMX-Med12, and were grown in the absence or presence of 50 nM Gefitinib. Cells were then fixed, stained and photographed after 2 weeks (untreated) or 3 weeks (EGFR inhibitor treatment). (B) The MED12/Med12 protein levels in PC9 cells (untreated) described in FIG. 3A. (C and D) The endogenous MED12 mRNA (C) and the exogenous Med12 mRNA were measured by qRT-PCR.

FIG. 7 shows that suppression of MED12 confers drug resistance to EGFR inhibitors in H3255 EGFR mutant cells. (A) H3255 cells expressing shRNAs targeting MED12 are resistant to EGFR inhibitors. The functional phenotypes of shMED12 vectors are indicated by the colony formation assay in 25 nM Gefitnib or 25 nM Erlotinib. The cells were fixed, stained and photographed after 2 weeks (untreated) or 4 weeks (EGFR inhibitors treatment). (B) The knockdown ability of each of the shRNAs was measured by examining the MED12 mRNA levels by qRT-PCR. Error bars denote standard deviation (SD).

FIG. 8 shows that suppression of ARID1A confers drug resistance to EGFR and MET inhibitors in NSCLC cells with mutant EGFR or MET amplification. (A) PC9 cells expressing shRNAs targeting ARID1A are resistant to EGFR inhibitor. The functional phenotypes of shARID1A vectors are indicated by the colony formation assay in 25 nM Gefitinib. The cells were fixed, stained and photographed after 2 weeks (untreated) or 4 weeks (EGFR inhibitor treatment). (B) The ARID1A mRNA levels for the cells described in FIG. 8A were measured by qRT-PCR. Error bars denote standard deviation (SD). (C) H1993 cells expressing shRNAs targeting ARID1A are resistance to MET inhibitor. The functional phenotypes of shARID1A vectors are indicated by the colony formation assay in 200 nM Crizotinib. The cells were fixed, stained and photographed after 2 weeks (untreated) or 4 weeks (MET inhibitor treatment). (D) The ARID1A mRNA levels for the cells described in FIG. 8C were measured by qRT-PCR. Error bars denote standard deviation (SD).

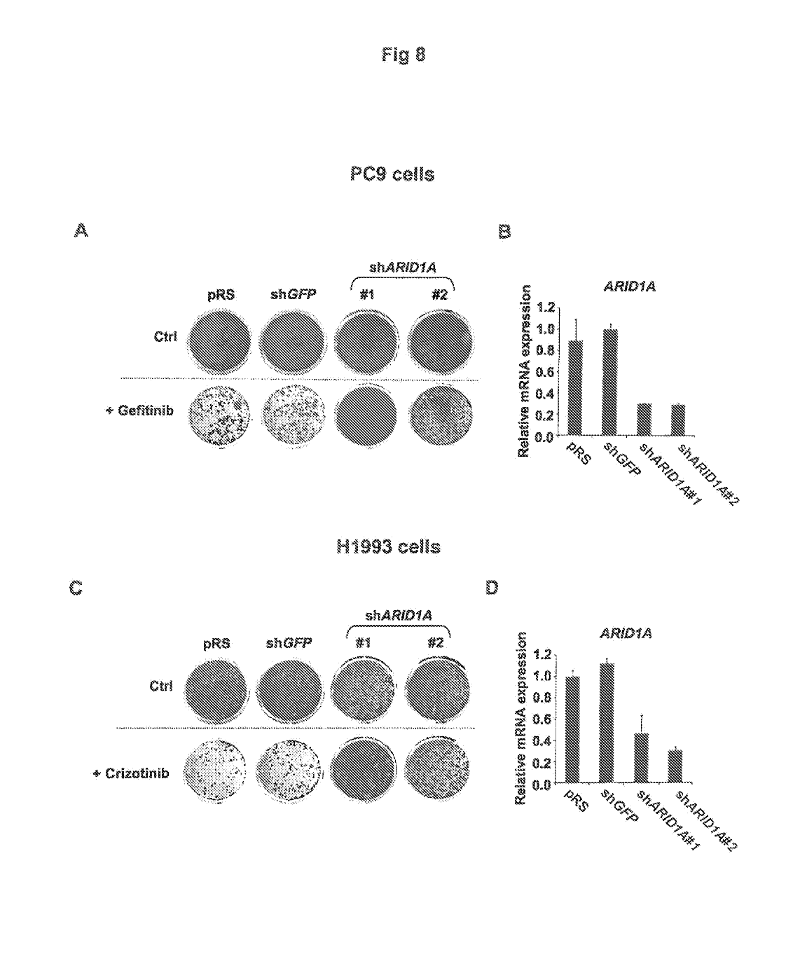

FIG. 9 shows that restoration of SMARCE1 reverses the resistance to EGFR inhibitor driven by SMACRE1 knockdown in PC9 EGFR mutant cells. (A) Ectopic expression of SMARCE1-ND that cannot be targeted by shSMARCE1 vectors re-sensitizes the otherwise resistant SMARCE1 knockdown cells to EGFR inhibitor. PC9 cells expressing pRS control or shSMARCE1 vectors were retrovirally infected with viruses containing pMX or pMX-SMARCE1-ND, and were grown in the absence or presence of 50 nM Gefitinib. Cells were then fixed, stained and photographed after 2 weeks (untreated) or 4 weeks (EGFR inhibitor treatment). (B) The SMARCE1 protein levels in PC9 cells (untreated) described in FIG. 9A. (C and D) The endogenous SMARCE1 mRNA was measured by qRT-PCR using a 3′ UTR specific primer set (C) and the total SMARCE1 mRNA was measured by qRT-PCR using an ORF specific primer set.

FIG. 10 shows that restoration of SMARCE1 reverses the resistance to MET inhibitor driven by SMACRE1 knockdown in H1993 MET amplified cells. (A) Ectopic expression of SMARCE1-ND that cannot be targeted by shSMARCE1 vectors re-sensitizes the otherwise resistant SMARCE1 knockdown cells to MET inhibitor. H1993 cells expressing pRS control or shSMARCE1 vectors were retrovirally infected with viruses containing pMX or pMX-SMARCE1-ND, and were grown in the absence or presence of 200 nM Crizotinib. Cells were then fixed, stained and photographed after 2 weeks (untreated) or 4 weeks (MET inhibitor treatment). (B) The SMARCE1 protein levels in H1993 cells (untreated) described in FIG. 10A. (C and D) The endogenous SMARCE1 mRNA was measured by qRT-PCR using a 3′ UTR specific primer set (C) and the total SMARCE1 mRNA was measured by qRT-PCR using an ORF specific primer set.

FIG. 11 shows that restoration of SMARCE1 reverses the resistance to MET inhibitor driven by SMACRE1 knockdown in EBC1 MET amplified cells. (A) Ectopic expression of SMARCE1-ND that cannot be targeted by shSMARCE1 vectors re-sensitizes the otherwise resistant SMARCE1 knockdown cells to MET inhibitor. EBC1 cells expressing pRS control or shSMARCE1 vectors were retrovirally infected with viruses containing pMX or pMX-SMARCE1-ND, and were grown in the absence or presence of 200 nM Crizotinib. Cells were then fixed, stained and photographed after 2 weeks (untreated) or 4 weeks (MET inhibitor treatment). (B) The SMARCE1 protein levels in HI 993 cells (untreated) described in FIG. 11A. (C and D) The endogenous SMARCE1 mRNA was measured by qRT-PCR using a 3′ UTR specific primer set (C) and the total SMARCE1 mRNA was measured by qRT-PCR using an ORF specific primer set.

FIG. 12 depicts a RAS-GAP RNAi screen that identifies DAB2IP and NF as critical determinants of drug sensitivity to EGFR inhibitors in EGFR mutant NSCLC cells. PC9 cells expressing controls (pLKO or shGFP) or 14 pools of shRNA vectors targeting each RAS-GAP were grown in the absence or presence of 50 nM Gefitinib or Elortinib. Cells were then fixed, stained and photographed after 2 weeks (untreated) or 4 weeks (EGFR inhibitors treatment).

FIG. 13 shows that suppression of DAB2IP confers drug resistance to EGFR inhibitors in PC9 EGFR mutant cells. (A) Validation of independent shRNAs (in pLKO vector) targeting DABP2IP in PC9 cells. The functional phenotypes of non-overlapping shDABP2IP vectors are indicated by the colony formation assay in 50 nM Gefitinib or Elortinib. The cells were fixed, stained and photographed after 2 weeks (untreated) or 4 weeks (EGFR inhibitors treatment). (B) The knockdown ability of each of the shRNAs was measured by examining the DAB21IP mRNA levels by qRT-PCR. Error bars denote standard deviation (SD). (C) Western blotting analysis of PC9 cells expressing controls (pLKO or shGFP) or shRNAs targeting DAB2IP treated with vehicle control or 25 nM Gefitinib for 8 hours.

FIG. 14 shows that suppression of NF1 confers drug resistance to EGFR inhibitors in PC9 EGFR mutant cells. (A) Validation of independent shRNAs (in pLKO vector) targeting NF1 in PC9 cells. The functional phenotypes of non-overlapping shNF1 vectors are indicated by the colony formation assay in 50 nM Gefitinib or Elortinib. The cells were fixed, stained and photographed after 2 weeks (untreated) or 4 weeks (EGFR inhibitors treatment). (B and C) The knockdown ability of each of the shRNAs was measured by examining the NF1 mRNA levels by qRT-PCR (B) and the NF 1 protein levels by western blotting (C). Error bars denote standard deviation (SD).

FIG. 15 shows that suppression of MED12 and SMARCE1 leads to elevated phospho-AKT levels. (B) SMARCE1^{KD} cells have elevated phospho-AKT levels in EML4-ALK cells. H3122 cells expressing controls (pRS or shGFP) or shSMARCE1 vectors were gown in the absence or presence of 20 nM NVP-TAE684 for 24 hours and the cell lysates were harvested for western blotting analysis. (C) MED12^{KD} cells have elevated phospho-AKT levels in EGFR mutant cells. PC9 cells expressing controls (pRS or shGFP) or shSMARCE1 vectors were gown in the absence or presence of 25 nM Gefitinib for 8 hours and the cell lysates were harvested for western blotting analysis.

FIG. 16 shows that ARID1A (SMARCF1) loss also confers resistance to targeted cancer therapeutics in breast cancer. (A) Overview of shRNA bar code screens performed in breast cancer cell lines, the drugs used in the screen and the validated outliers from the screen: genes whose suppression confers resistance to the indicated drug. (B) Validation of independent shRNAs (in pLKO vector) targeting ARID1A in T47D breast cancer cells. TIhe functional phenotypes of non-overlapping shARID1A vectors are indicated by the colony formation assay in 10 and 20 nM small molecule mTOR inhibitor AZD8055. The cells were fixed, stained and photographed after 2 weeks (untreated) or 4 weeks (inhibitor treatment). Ectopic expression of a mutant of cyclin DI (pBpDITA) served as a control in the experiment. (C) Validation of independent shRNAs (in pLKO vector) targeting ARID1A in MCF7 breast cancer cells. The functional phenotypes of non-overlapping shARIDJA vectors are indicated by the colony formation assay in 20 and 40 nM small molecule mTOR inhibitor AZD8055. The cells were fixed, stained and photographed after 2 weeks (untreated) or 4 weeks (inhibitor treatment). (D). Quantification of ARID1A mRNA levels by QRT-PCR after knockdown of ARID1A in MCF7 breast cancer cells.

FIG. 17 shows that ARID1A (SMARCF1) loss also confers resistance to trastuzumab in HER2-amplified breast cancer, shRNA mediated knockdown of ARID1A was induced in BT474 breast cancer cells. The functional phenotypes of non-overlapping shARID1A vectors are indicated by the colony formation assay in 25 nM small molecule mTOR inhibitor AZD8055 or 2 ug/ml of trastuzumab (Herceptin). The cells were fixed, stained and photographed after 2 weeks (untreated) or 4 weeks (inhibitor treatment).

FIG. 18 shows that ARID1A loss leads to activation of PI3K/mTOR signaling.

### DETAILED DESCRIPTION

The instant invention provides methods and related compositions pertaining to the identification of a tumor that will be resistant to treatment by a certain compound or class of compounds. In certain embodiments, the invention provides one or more markers for resistance to anticancer treatment in a patient. In some embodiments, the marker is a MEDIATOR complex and/or SWI/SNF complex gene.

Examples of MEDIATOR complex genes that may serve as a marker for resistance to anticancer treatment in a patient as described herein include MED22, MED11, MED17, MED20, MED30, MED19, MED18, MED8, MED6, MED28, MED9, MED21, MED4, MED7, MED31, MED10, MED1, MED26, MED2, MED3, MED25, MED23, MED5, MED14, MED16, MED15, CycC, CDK8, MED13, MED12, MED13L, and MED12L (see e.g., MED12L Gene ID: 116931 available from the National Center for Biotechnology Information (NCBI) website). See, e.g., Malik, S, Roeder, R G, “The metazoan Mediator co-activator complex as an integrative hub for transcriptional regulation” Nat Rev Genet. (2010) 11(11):761-72.

Examples of SWI/SNF complex genes that may serve as a marker for resistance to anticancer treatment in a patient as described herein include ARID1A, ARID1B, ARID2, SMARCA2, SMARCA4, PBRM1, SMARCC2, SMARCC1, SMARCD1, SMARCD2, SMARCD3, SMARCE1, ACTL6A, ACTL6B, and SMARCB1. See, e.g., Reisman, D et al. “The SWI/SNF complex and cancer” Oncogene. (2009) 28(14):1653-68.

In some embodiments, the invention provides methods whereby measurement of reduced expression of a MEDIATOR complex and/or SWI/SNF complex gene in one or more cancer cells ofa patient identifies these cancer cells as cells that may be resistant to treatment by one or more receptor tyrosine kinase (RTK) inhibitors. RTKs are involved in a number of diverse physiological processes, including proliferation and differentiation, cell survival and metabolism, cell migration, and cell-cycle control (see, e.g., Lemmon, M A, Schlessinger, J “Cell Signaling by Receptor Tyrosine Kinases” Cell (2010) 141:1117-1134).

In certain embodiments, identification of a reduced expression of a MEDIATOR complex and/or SWI/SNF complex gene in one or more cancer cells of a patient is indicative that the one or more cancer cells will be resistant to treatment by a compound or class of compounds, such as one or more receptor tyrosine kinase inhibitor compounds. Examples of RTK inhibitor compounds that cells expressing a reduced level of a MEDIATOR complex and/or SWI/SNF complex gene may be resistant to include gefitinib, erlotinib, EKB-569, lapatinib, CI-1033, cetuximab, panitumumab, PKI-166, AEE788, sunitinib, sorafenib, dasatinib, nilotinib, pazopanib, vandetaniv, cediranib, afatinib, motesanib, CUDC-101, and imatinib mesylate. Other RTK inhibitors that cells expressing a reduced level of a MEDIATOR complex and/or SWI/SNF complex gene may be resistant to include the Alk-1 inhibitors crizotinib, ASP-3026, LDK378, AF802, and CEP37440.

In certain embodiments, identification of a reduced expression of a MEDIATOR complex and/or SWI/SNF complex gene in one or more cancer cells of a patient is indicative that the one ore more cancer cells will be resistant to treatment by one or more Akt activation inhibitors. Examples of Akt activation inhibitor compounds that cells expressing a reduced level of a MEDIATOR complex and/or SWI/SNF complex gene may be resistant to include compounds that inhibit the activity of an RTK signaling protein upstream of Akt. In certain embodiments, the compound that inhibits the activity of an RTK signaling protein upstream of Akt inhibits a direct activator of Akt. In other embodiments, the compound that inhibits the activity of an RTK signaling protein upstream of Akt inhibits an indirect activator of Akt. In certain embodiments, the inhibitor of Akt is an inhibitor of phosphatidylinositol-3-kinase (PI3K). Examples of PI3K inhibitors include NVP-BKM120, XL147 (SAR245408), PX-866, GDC-0941, CAL-101, CNX-1351, ETP-46992, RP-5002, XL-499, and ONC-201.

In certain embodiments, identification of a reduced expression of a MEDIATOR complex and/or SWI/SNF complex gene in one or more cancer cells of a patient is indicative that the one or more cancer cells will be resistant to treatment by a compound or class of compounds, such as one or more receptor mTOR inhibitor compounds. Examples of mTOR inhibitor compounds include rapamycin/sirolimus, temsirolimus, everolimus, PP242, PP30, INK128, WYE-600, WYE-687, WYE-354, INK128, AZD8055, Torin-1, AZD2014, ridaforolimus, OSI-027, NV-128, NV-344, AP-23675, AP-23841, AP-24170, and TAFA-93. In yet other embodiments, the compound or class of compounds inhibit both PI3K and mTOR. Examples of such compounds include the dual PI3K and mTOR inhibitors BEZ235, BGT226, SF1126, GSK1059615, PKI-402, PX866, GDC0941/GDC080, BKM120, NVP-BEZ235, NVP-BGT226, PF-04691502, PF-04979064, PF-05177624, PF-05197281, PF-05212384, XL147, XL765, EXEL-1229, EXEL-2400, EXEL-3751, EXEL-4251, PWT-33597, and SB2343.

In certain embodiments, the prognostic methods and compositions of the instant invention predict resistance to anticancer treatment to a combination of chemotherapeutic agents, wherein the at least two chemotherapeutic agents are administered at the same time and/or sequentially. In further embodiments, the invention provides methods wherein a measurement of reduced expression of a MEDIATOR complex and/or SWI/SNF complex and/or RAS-GAP gene in or one or more cancer cells of a tumor of a patient identifies the tumor as one that may be resistant to treatment by a combination of at least two Akt activation inhibitors. In other embodiments, the tumor is one that may be resistant to treatment by a combination of at least two compounds that activate one or more proteins upstream of Akt that inactivates Akt signaling.

In some embodiments, the markers of the instant invention enable the detection of resistance to anticancer treatment in a patient in combination with one or more known markers of hypersensitivity to a chemotherapeutic agent or class of agents. In certain embodiments, expression levels of one or more MEDIATOR complex and/or SWI/SNF complex genes (e.g., MED12, SMARCE1, and/or ARIDA1) are measured in one or more cancer cells of a patient in combination with an array profile, such as a CGH (comparative genomic hybridization) array analysis.

In certain embodiments, the invention provides methods and compositions for identifying a cancer patient who would likely not benefit from a certain chemotherapeutic treatment. For example, an aspect of the invention is a method of screening cancer patients to determine those cancer patients more likely to benefit from a particular chemotherapy, such as PI3K and/or mTOR inhibitor chemotherapy, comprising obtaining a sample of genetic material from a tumor of the patient; and assaying for the presence of a genotype in the patient that is associated with resistance to the particular chemotherapy, the genotype characterized by an inactivating mutation in one or more MEDIATOR complex and/or SWI/SNF complex genes. In some embodiments, the genotype is further characterized by an inactivating mutation in one or more known markers for chemotherapeutic resistance. In some embodiments, the genetic material is nucleic acid that is characterized by a reduced expression (e.g., reduced mRNA levels) of one or more MEDIATOR complex and/or SWI/SNF complex genes. In further embodiments, reduced mRNA levels are assessed by the evaluating the corresponding cDNA.

In a particular embodiment, the instant invention provides methods and compositions for the identification of a lung cancer patient who would likely not benefit from RTK inhibitor chemotherapy (e.g., the patient will be recurrence-free for a period of time less than a patient undergoing the same chemotherapy). In another embodiment, the instant invention provides methods and compositions for the identification of a breast cancer patient who would likely not benefit from treatment with Herceptin (e.g., the breast cancer cells in the patient would likely be resistant to Herceptin treatment). In some embodiments, the methods of the instant invention predict whether a chemotherapeutic agent or other compound is likely to be cytotoxic to one or more cancer cells.

Cancers for which the prognostic methods and compositions of the instant invention may provide predictive results for resistance to anticancer treatment include cancers such as breast cancer (e.g., BRCA-1 deficient, stage-III HER2-negative, ER and metastatic breast cancers), ovarian cancer (e.g., BRCA-1 deficient, epithelial ovarian cancer), lung cancer (e.g., non-small-cell lung cancer or small cell lung cancer, metastatic non-small cell lung cancer), liver cancer (e.g., hepatocellular carcinoma), bladder cancer (e.g., transitional cell carcinoma of the bladder), and colorectal cancer (e.g., advanced (non-resectable locally advanced or metastatic) colorectal cancer). Other cancers for which the methods and compositions of the invention may provide predictive results for resistance to anticancer treatment include cervical cancer (e.g., recurrent and stage IVB), mesothelioma, solid tumors (e.g., advanced solid tumors), renal cell carcinoma (e.g., advanced renal cell carcinoma), esophageal cancer, stomach cancer, head and neck cancers (e.g., metastatic squamous cell carcinoma of the head and neck (SCCHN), squamous cell carcinoma, laryngeal cancer, hypopharyngeal cancer, oropharyngeal cancer, and oral cavity cancer), sarcoma, prostate cancer (e.g., hormone refractory prostate cancer), melanoma, thyroid cancer (e.g., papillary thyroid cancer), brain cancer, adenocarcinoma, subependymal giant cell astrocytoma, endometrial cancer, neuroblastoma, glioma, glioblastoma, and other tumors that have metastasized to the brain, lymphoma, and hematological cancers.

In some embodiments, the cancer is one in which one or more RTK inhibitor drugs are employed either alone or in combination with other chemotherapeutic agents as a part of an anticancer treatment regimen. In other embodiments, the cancer is one in which one or more RTK inhibitor drugs are employed either alone or in combination with additional treatment regimens, such as surgical procedures, radiation, and/or other anticancer treatments. In certain embodiments, the cancer is one in which one or more RTK inhibitor agents are used as a first-line form of treatment.

In certain embodiments, the instant invention relates to methods and compositions encompassing the detection of expression levels of a MEDIATOR complex and/or SWI/SNF complex and/or RAS-GAP gene in one or more cells of a subject. Typically, the subject is a human patient who has or is suspected of having at least one type of cancer, and the expression levels of the MEDIATOR complex and/or SWI/SNF complex and/or RAS-GAP gene are detected in a sample of one or more cells, typically one or more tumor cells, from the human patient, which are then compared with the expression levels of the MEDIATOR complex and/or SWI/SNF complex gene and/or RAS-GAP gene in a control sample. A control sample will generally be one in which the MEDIATOR complex and/or SWI/SNF complex and/or RAS-GAP gene expression levels are known and correlated with resistance to anticancer treatment to a certain drug or group of drugs. In some embodiments, the control sample is one in which the MEDIATOR complex and/or SWI/SNF complex and/or RAS-GAP gene expression levels are known and correlated with a lack of resistance to anticancer treatment to a certain drug or group of drugs. In certain embodiments, the MEDIATOR complex and/or SWI/SNF complex and/or RAS-GAP gene expression levels in one or more tumor cells of a patient are compared with the expression levels in one or more normal cells of the patient, wherein a reduced expression in the one or more tumor cells in comparison to the one or more normal cells of the patient are predictive of resistance to anticancer treatment to a certain drug or group of drugs. In some embodiments, the control sample is one in which the MEDIATOR complex and/or SWI/SNF complex and/or gene expression levels are known and correlated with a lack of resistance to anticancer treatment to a certain drug or group of drugs. In some embodiments, more than one control sample is used for comparative purposes with the test sample from the subject. In certain embodiments, the expression levels of a MEDIATOR complex gene are detected. In other embodiments, the expression levels of a SWI/SNF complex gene are detected. In yet other embodiments, the expression levels of a RAS-GAP gene are detected.

In certain embodiments, the invention relates to a method for predicting a breast cancer patient's response to RTK inhibitor drug chemotherapy, such as Herceptin treatment. Typically, the breast cancer patient has not yet received RTK inhibitor drug chemotherapy. In further embodiments, a sample of the breast cancer cells from the patient is analyzed for the levels of expression of a MEDIATOR complex and/or SW/SNF complex gene, such as MED12, SMARCE1, and/or ARIDA1, and or a RAS-GAP gene, such as DAB2IP, NF1, and/or RASAL3. If expression levels of the MEDIATOR complex and/or SWI/SNF complex gene (e.g., MED12, SMARCE1, and/or ARIDA1) and/or RAS-GAP gene (e.g., DAB2IP, NF1, and/or RASAL3) are low compared to expression levels in normal breast tissue, then the breast cancer cells in the patient are likely resistant to RTK inhibitor anticancer treatment.

In certain embodiments, the expression level of the MEDIATOR complex and/or SWI/SNF complex gene, such as MED12, SMARCE1, and/or ARIDA1, and/or RAS-GAP gene, such as DAB2JP, NF1, and/or RASAL3 in cancer tissue is lower than the expression level of the gene in normal tissue. In predicting resistance to anticancer treatment of a tumor, cut-off levels of expression may be determined empirically for the subject cancer for which resistance to anticancer treatment is being assessed.

In other embodiments, the instant invention relates to methods and compositions encompassing the detection of one or more inactivating mutations in a MEDIATOR complex and/or SWI/SNF complex and/or RAS-GAP gene in one or more cells of a subject. Typically, the subject is a human patient who has or is suspected of having at least one type of cancer, and the nucleic acid of the MEDIATOR complex and/or SWI/SNF complex and/or RAS-GAP are isolated from a sample of one or more cells, typically one or more tumor cells, from the human patient, which are then compared with the nucleic acid of the MEDIATOR complex and/or SWI/SNF complex and/or RAS-GAP in a control sample. A control sample will generally be one in which the MEDIATOR complex and/or SWI/SNF complex and/or RAS-GAP nucleic acid sequences are known and correlated with resistance to anticancer treatment to a certain drug or group of drugs. In some embodiments, the control sample is one in which the MEDIATOR complex and/or SWI/SNF complex and/or RAS-GAP nucleic acid sequences are known and correlated with a lack of resistance to anticancer treatment to a certain drug or group of drugs. In some embodiments, more than one control sample is used for comparative purposes with the test sample from the subject. In certain embodiments, the inactivating mutation is a point mutation. In some embodiments, the inactivating mutation is a hypomorphic mutation. In other embodiments, the inactivating mutation is a gene deletion. In yet other embodiments, the inactivating mutation is an amplification.

In some embodiments, the instant invention relates to methods and compositions encompassing evaluating the protein activity and/or sequence and/or posttranslational modification state of one or more RAS-GAP proteins and/or proteins in a MEDIATOR complex and/or SWI/SNF complex in one or more cells of a subject. Typically, the subject is a human patient who has or is suspected of having at least one type of cancer, and the RAS-GAP protein and/or protein of the MEDIATOR complex and/or SWI/SNF complex is isolated from a sample of one or more cells, typically one or more tumor cells, from the human patient, which are then compared with the RAS-GAP protein and/or protein of the MEDIATOR complex and/or SWI/SNF complex in a control sample. A control sample will generally be one in which the RAS-GAP protein and/or MEDIATOR complex and/or SWI/SNF complex protein sequences and/or activity and/or posttranslational modification state are known and correlated with resistance to anticancer treatment to a certain drug or group of drugs. In some embodiments, the control sample is one in which the RAS-GAP protein and/or MEDIATOR complex and/or SWI/SNF complex protein sequences and/or activity and/or posttranslational modification state are known and correlated with a lack of resistance to anticancer treatment to a certain drug or group of drugs.

Evaluation of protein activity includes assaying the enzymatic activity of the protein. In certain embodiments, the posttranslational modification status of the protein is assessed. In further embodiments, one or more posttranslational modifications (or lack thereof) is associated with protein dysfunction, such as reduced enzymatic activity by the protein. In some embodiments, the RAS-GAP and/or MEDIATOR complex and/or SWI/SNF complex protein in one or more cells of a subject is dysfunctional, and this dysfunction is indicative of resistance to one or more anticancer treatments. Examples of protein dysfunction include reduced or no enzymatic and/or binding activity of the protein; reduced or no protein expression; and/or improper protein modification, such as phosphorylation that results in inactivity of the protein.

The terms “marker” and “biomarker” are used interchangeably herein and refer to a gene, protein, or fragment thereof, the expression or level or activity of which changes between certain conditions. Where the expression or level or activity of the gene, protein, or fragment thereof correlates with a certain condition, the gene, protein, or fragment thereof is a marker for that condition.

“Resistant,” “resistance,” or “resistance to anticancer treatment” in the context of treatment of a cancer cell with a chemotherapeutic agent or other compound means that the chemotherapeutic agent or other compound is not likely to have an optimal effect on the cancer cell. In some embodiments, the compound is not likely to have any effect on the cancer cell. In certain embodiments, the effect of a compound on one or more cancer cells is reduced. In certain further embodiments, a tumor is likely to be less sensitive to a compound but not completely resistant to it. In certain embodiments, the compound is not likely to be cytotoxic to the cancer cell. In some embodiments, the compound is not cytotoxic to the cancer cell.

By “primary resistance” with regard to one or more cancer cells in a patient is meant cells that are naïve for anticancer treatment. For example, a tumor that demonstrates primary resistance to an anticancer treatment includes one that has never been treated with the anticancer drug or drugs but demonstrates or is predicted to demonstrate resistance to the anticancer drug or drugs once treatment has begun.

By “secondary resistance” with regard to one or more cancer cells in a patient is meant cells that have acquired resistance to an anticancer treatment. For example, a tumor that demonstrates secondary resistance to an anticancer treatment includes one that has been treated for a prolonged period of time with one or more anticancer drugs but resistance arises to the one or more anticancer drugs after treatment.

By “inactivating mutation” is meant a mutation in, for example, a nucleic acid that encodes a protein that is inactive. This includes, for example, mutations that result in the loss of protein expression and/or activity and includes genetic mutations such as point mutations, translocations, amplifications, deletions (including whole gene deletions), and hypomorphic mutations (e.g., where an altered gene product possesses a reduced level of activity or where the wild-type gene product is expressed at a reduced level). “Inactivating mutation” also includes biomarker dysfunctions due to post-translational protein regulation, for example, where a protein biomarker is inactive or exhibits impaired activity due to, for example, one or more posttranslational modifications, such as phosphorylation that results in protein inactivity.

The term “biomarker dysfunction” with regard to a protein or protein fragment refers to dysfunction of the protein or fragment thereof as a result of improper regulation at the posttranslational level, such as, for example, phosphorylation that results in protein inactivity.

By “MEDIATOR complex gene” is meant any gene encoding for a protein of the MEDIATOR complex.

By “reference MEDIATOR complex gene” is meant a MEDIATOR complex gene in a control sample, e.g., a normal sample such as a non-cancerous tissue sample. Typically, the expression levels of a reference MEDIATOR complex gene serve as a reference for comparative purposes with the levels of expression of the same MEDIATOR complex gene in a different sample, typically a test sample, such as a lung tumor sample.

By “SWI/SNF complex gene” is meant any gene encoding for a protein of the SWI/SNF complex.

By “reference SWI/SNF complex gene” is meant a SWI/SNF complex gene in a control sample, e.g., a normal sample such as a non-cancerous tissue sample. Typically, the expression levels of a reference SWI/SNF complex gene serve as a reference for comparative purposes with the levels of expression of the same SWI/SNF complex gene in a different sample, typically a test sample, such as a lung tumor sample.

By “RAS-GAP gene” is meant any gene encoding for a RAS-GAP protein.

By “reference RAS-GAP gene” is meant a RAS-GAP gene in a control sample, e.g., a normal sample such as a non-cancerous tissue sample. Typically, the expression levels of a reference RAS-GAP gene serve as a reference for comparative purposes with the levels of expression of the same RAS-GAP gene in a different sample, typically a test sample, such as a lung tumor sample.

By “interact directly” is meant that a protein or other molecular compound binds and/or enzymatically interacts with a target protein.

By “interact indirectly” is meant that a protein or other molecular compound binds and/or enzymatically interacts with a cellular protein or other molecular compound that may itself interact with a second cellular protein and so forth until a final cellular protein interacts directly with a target protein. This includes any upstream activators of a target protein, such as Akt, in a signaling cascade, such as a receptor tyrosine kinase signaling cascade.

As used herein, the terms “drug,” “agent,” and “compound,” either alone or together with “chemotherapeutic” or “chemotherapy,” encompass any composition of matter or mixture which provides some pharmacologic effect that can be demonstrated in-vivo or in vitro. This includes small molecules, antibodies, microbiologicals, vaccines, vitamins, and other beneficial agents. As used herein, the terms further include any physiologically or pharmacologically active substance that produces a localized or systemic effect in a patient.

The term “nucleic acid” encompasses DNA, RNA (e.g., mRNA, tRNA), heteroduplexes, and synthetic molecules capable of encoding a polypeptide and includes all analogs and backbone substitutes such as PNA that one of ordinary skill in the art would recognize as capable of substituting for naturally occurring nucleotides and backbones thereof. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences which encode a particular amino acid sequence.

Unless otherwise indicated, nucleic acids are written left to right in 5′ to 3′ orientation; amino acid sequences are written left to right in amino to carboxy orientation, respectively.

“Antisense” nucleic acids are DNA or RNA molecules that are complementary to at least a portion ofa specific mRNA molecule (Weintraub, Scientific American 262 40, 1990). In the cell, the antisense nucleic acids hybridize to the corresponding mRNA, forming a double-stranded molecule. This interferes with the translation of the mRNA since the cell will not translate an mRNA that is double-stranded. Antisense oligomers of at least about 15, about 20, about 25, about 30, about 35, about 40, or of at least about 50 nucleotides are preferred, since they are easily synthesized and are less likely to cause non-specific interference with translation than larger molecules. The use of antisense methods to inhibit the in vitro translation of genes is well known in the art (Marcus-Sakura Anal. Biochem. 172: 289, 1998).

Short double-stranded RNAs (dsRNAs; typically <30 nucleotides) can be used to silence the expression of target genes in animals and animal cells. Upon introduction, the long dsRNAs enter the RNA interference (RNAi) pathway which involves the production of shorter (20-25 nucleotide) small interfering RNAs (siRNAs) and assembly of the siRNAs into RNA-induced silencing complexes (RISCs). The siRNA strands are then unwound to form activated RISCs, which cleave the target RNA. Double stranded RNA has been shown to be extremely effective in silencing a target RNA.

General methods of using antisense, ribozyme technology and RNAi technology, to control gene expression, or of gene therapy methods for expression of an exogenous gene in this manner are well known in the art. Each of these methods utilizes a system, such as a vector, encoding either an antisense or ribozyme transcript. The term “RNAi” stands for RNA interference. This term is understood in the art to encompass technology using RNA molecules that can silence genes. See, for example, McManus, et al. Nature Reviews Genetics 3: 737, 2002. In this application, the term “RNAi” encompasses molecules such as small interfering or short interfering RNA (siRNA), small hairpin or short hairpin RNA (shRNA), microRNAs, and small temporal RNA (stRNA). Generally speaking, RNA interference results from the interaction of double-stranded RNA with genes.

The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. In certain embodiments, siRNA molecules are 12-28 nucleotides long, more preferably 15-25 nucleotides long, still more preferably 19-23 nucleotides long and most preferably 21-23 nucleotides long. In certain embodiments, preferred siRNA molecules are 12, 13, 14, 15, 16, 17, 18, 19, 20, 21, 22, 23, 24, 25, 26, 27 28 or 29 nucleotides in length.

As used herein, the term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter code for amino acid residues are used herein.

As used herein, a “synthetic” molecule is produced by in vitro chemical or enzymatic synthesis rather than by an organism.

As used herein, the term “expression” refers to the process by which a polypeptide is produced based on the nucleic acid sequence of a gene. The process includes both transcription and translation. The term “expression” also includes the protein product of a translated mRNA. The term “expression” as it refers to protein includes both protein levels and protein activity (e.g., protein binding, enzymatic activity, etc.). The term “expression” also refers to the transcription of non-translated nucleic acid (e.g., non-coding mRNA).

A “gene” refers to the DNA segment encoding a polypeptide or RNA.

By “homolog” is meant an entity having a certain degree of identity with the subject amino acid sequences and the subject nucleotide sequences. As used herein, the term “homolog” covers identity with respect to structure and/or function, for example, the expression product of the resultant nucleotide sequence has the enzymatic activity of a subject amino acid sequence. With respect to sequence identity, preferably there is at least 70%, 75%, 80%, 81%, 82%, 83%, 84%, 85%, 86%, 87%, 88%, 89%, 90%, 91%, 92%, 93%, 94%, 95%, 96%, 97%, 98%, or even 99% sequence identity. These terms also encompass allelic variations of the sequences. The term, homolog, may apply to the relationship between genes separated by the event of speciation or to the relationship between genes separated by the event of genetic duplication.

Relative sequence identity can be determined by commercially available computer programs that can calculate % identity between two or more sequences using any suitable algorithm for determining identity, using, for example, default parameters. A typical example of such a computer program is CLUSTAL. Advantageously, the BLAST algorithm is employed, with parameters set to default values. The BLAST algorithm is described in detail on the National Center for Biotechnology Information (NCBI) website.

The homologs of the peptides as provided herein typically have structural similarity with such peptides. A homolog of a polypeptide includes one or more conservative amino acid substitutions, which may be selected from the same or different members of the class to which the amino acid belongs.

In one embodiment, the sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

The present invention also encompasses conservative substitution (substitution and replacement are both used herein to mean the interchange of an existing amino acid residue with an alternative residue) that may occur e.g., like-for-like substitution such as basic for basic, acidic for acidic, polar for polar, etc. Non-conservative substitution may also occur e.g., from one class of residue to another or alternatively involving the inclusion of unnatural amino acids such as omithine (hereinafter referred to as Z), diaminobutyric acid ornithine (hereinafter referred to as B), norleucine ornithine (hereinafter referred to as O), pyriylalanine, thienylalanine, naphthylalanine and phenylglycine. Conservative substitutions that may be made are, for example, within the groups of basic amino acids (Arginine, Lysine and Histidine), acidic amino acids (glutamic acid and aspartic acid), aliphatic amino acids (Alanine, Valine, Leucine, Isoleucine), polar amino acids (Glutamine, Asparagine, Serine, Threonine), aromatic amino acids (Phenylalanine, Tryptophan and Tyrosine), hydroxyl amino acids (Serine, Threonine), large amino acids (Phenylalanine and Tryptophan) and small amino acids (Glycine, Alanine).

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enymology: DNA Structure Part A: Synthesis and Physical Analysis of DNA Methods in Enzymology, Academic Press. Each of these general texts is herein incorporated by reference.

### Methods of Detecting Expression Levels

There are many methods known in the art for determining the genotype of a patient. Any method for determining genotype can be used for determining genotypes in the present invention. Such methods include, but are not limited to, amplimer sequencing, DNA sequencing, fluorescence spectroscopy, fluorescence resonance energy transfer (or “FRET”)-based hybridization analysis, high throughput screening, mass spectroscopy, nucleic acid hybridization, polymerase chain reaction (PCR), RFLP analysis and size chromatography (e.g., capillary or gel chromatography), all of which are well known to one of ordinary skill in the art.

Many methods of sequencing genomic DNA are known in the art, and any such method can be used, see for example Sambrook et al., Molecular Cloning; A Laboratory Manual 2d ed. (1989). For example, a DNA fragment of interest can be amplified using the polymerase chain reaction or some other cyclic polymerase mediated amplification reaction. The amplified region of DNA can then be sequenced using any method known in the art. Advantageously, the nucleic acid sequencing is by automated methods (reviewed by Meldrum, Genome Res. September 2000; 10(9):1288-303, the disclosure of which is incorporated by reference in its entirety), for example using a Beckman CEQ 8000 Genetic Analysis System (Beckman Coulter Instruments, Inc.). Methods for sequencing nucleic acids include, but are not limited to, automated fluorescent DNA sequencing (see, e.g., Watts & MacBeath, Methods Mol Biol. 2001; 167:153-70 and MacBeath et al., Methods Mol Biol. 2001; 167:119-52), capillary electrophoresis (see, e.g., Bosserhoff et al., Comb Chem High Throughput Screen. December 2000; 3(6):455-66), DNA sequencing chips (see, e.g., Jain, Pharmacogenomics. August 2000; 1(3):289-307), mass spectrometry (see, e.g., Yates, Trends Genet. January 2000; 16(1):5-8), pyrosequencing (see, e.g., Ronaghi, Genome Res. January 2001; 11(1):3-11), and ultrathin-layer gel electrophoresis (see, e.g., Guttman & Ronai, Electrophoresis. December 2000; 21 (18):3952-64), the disclosures of which are hereby incorporated by reference in their entireties. The sequencing can also be done by any commercial company. Examples of such companies include, but are not limited to, the University of Georgia Molecular Genetics Instrumentation Facility (Athens, Ga.) or SeqWright DNA Technologies Services (Houston, Tex.).

Any one of the methods known in the art for amplification of DNA may be used, such as for example, the polymerase chain reaction (PCR), the ligase chain reaction (LCR) (Barany, F., Proc. Natl. Acad. Sci. (U.S.A.) 88:189-193 (1991)), the strand displacement assay (SDA), or the oligonucleotide ligation assay (“OLA”) (Landegren, U. et al., Science 241:1077-1080 (1988)). Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990)). Other known nucleic acid amplification procedures, such as transcription-based amplification systems (Malek, L. T. et al., U.S. Pat. No. 5,130,238; Davey, C. et al., European Patent Application 329,822; Schuster et al., U.S. Pat. No. 5,169,766; Miller, H. I. et al., PCT Application WO89/06700; Kwoh, D. et al., Proc. Natl. Acad. Sci. (U.S.A.) 86:1173 (1989); Gingeras, T. R. et al., PCT Application WO88/10315)), or isothermal amplification methods (Walker, G. T. et al., Proc. Natl. Acad. Sci. (U.S.A.) 89:392-396 (1992)) may also be used.

To perform a cyclic polymerase mediated amplification reaction according to the present invention, the primers are hybridized or annealed to opposite strands of the target DNA, the temperature is then raised to permit the thermostable DNA polymerase to extend the primers and thus replicate the specific segment of DNA spanning the region between the two primers. Then the reaction is thermocycled so that at each cycle the amount of DNA representing the sequences between the two primers is doubled, and specific amplification of gene DNA sequences, if present, results.

Any of a variety of polymerases can be used in the present invention. For thermocyclic reactions, the polymerases are thermostable polymerases such as Taq, KlenTaq, Stoffel Fragment, Deep Vent, Tth, Pfu, Vent, and UlTma, each of which are readily available from commercial sources. For non-thermocyclic reactions, and in certain thermocyclic reactions, the polymerase will often be one of many polymerases commonly used in the field, and commercially available, such as DNA pol 1, Klenow fragment, T7 DNA polymerase, and T4 DNA polymerase. Guidance for the use of such polymerases can readily be found in product literature and in general molecular biology guides.

Typically, the annealing of the primers to the target DNA sequence is carried out for about 2 minutes at about 37-55° C., extension of the primer sequence by the polymerase enzyme (such as Taq polymerase) in the presence of nucleoside triphosphates is carried out for about 3 minutes at about 70-75° C., and the denaturing step to release the extended primer is carried out for about 1 minute at about 90-95° C. However, these parameters can be varied, and one of skill in the art would readily know how to adjust the temperature and time parameters of the reaction to achieve the desired results. For example, cycles may be as short as 10, 8, 6, 5, 4.5, 4, 2, 1, 0.5 minutes or less.

Also, “two temperature” techniques can be used where the annealing and extension steps may both be carried out at the same temperature, typically between about 60-65° C., thus reducing the length of each amplification cycle and resulting in a shorter assay time.

Typically, the reactions described herein are repeated until a detectable amount of product is generated. Often, such detectable amounts of product are between about 10 ng and about 100 ng, although larger quantities, e.g. 200 ng, 500 ng, 1 mg or more can also, of course, be detected. In terms of concentration, the amount of detectable product can be from about 0.01 pmol, 0.1 pmol, 1 pmol, 10 pmol, or more. Thus, the number of cycles of the reaction that are performed can be varied, the more cycles are performed, the more amplified product is produced. In certain embodiments, the reaction comprises 2, 5, 10, 15, 20, 30, 40, 50, or more cycles.

For example, the PCR reaction may be carried out using about 25-50 μl samples containing about 0.01 to 1.0 ng of template amplification sequence, about 10 to 100 pmol of each generic primer, about 1.5 units of Taq DNA polymerase (Promega Corp.), about 0.2 mM dDATP, about 0.2 mM dCTP, about 0.2 mM dGTP, about 0.2 mM dTTP, about 15 mM MgCl.sub.2, about 10 mM Tris-HCl (pH 9.0), about 50 mM KCl, about 1 μg/ml gelatin, and about 10 μl/ml Triton X-100 (Saiki, 1988).

Those of ordinary skill in the art are aware of the variety of nucleotides available for use in the cyclic polymerase mediated reactions. Typically, the nucleotides will consist at least in part of deoxynucleotide triphosphates (dNTPs), which are readily commercially available. Parameters for optimal use of dNTPs are also known to those of skill, and are described in the literature. In addition, a large number of nucleotide derivatives are known to those of skill and can be used in the present reaction. Such derivatives include fluorescently labeled nucleotides, allowing the detection of the product including such labeled nucleotides, as described below. Also included in this group are nucleotides that allow the sequencing of nucleic acids including such nucleotides, such as chain-terminating nucleotides, dideoxynucleotides and boronated nuclease-resistant nucleotides. Commercial kits containing the reagents most typically used for these methods of DNA sequencing are available and widely used. Other nucleotide analogs include nucleotides with bromo-, iodo-, or other modifying groups, which affect numerous properties of resulting nucleic acids including their antigenicity, their replicatability, their melting temperatures, their binding properties, etc. In addition, certain nucleotides include reactive side groups, such as sulfhydryl groups, amino groups, N-hydroxysuccinimidyl groups, that allow the further modification of nucleic acids comprising them.

In certain embodiments, oligonucleotides that can be used as primers to amplify specific nucleic acid sequences of a gene in cyclic polymerase-mediated amplification reactions, such as PCR reactions, consist of oligonucleotide fragments. Such fragments should be of sufficient length to enable specific annealing or hybridization to the nucleic acid sample. The sequences typically will be about 8 to about 44 nucleotides in length, but may be longer. Longer sequences, e.g., from about 14 to about 50, are advantageous for certain embodiments.

In embodiments where it is desired to amplify a fragment of DNA, primers having contiguous stretches of about 8, 9, 10, 11, 12, 13, 14, 15, 16, 17, 18, 19, 20, 21, 22, 23, or 24 nucleotides from a gene sequence are contemplated.

As used herein, “hybridization” refers to the process by which one strand of nucleic acid base pairs with a complementary strand, as occurs during blot hybridization techniques and PCR techniques.

Whichever probe sequences and hybridization methods are used, one ordinarily skilled in the art can readily determine suitable hybridization conditions, such as temperature and chemical conditions. Such hybridization methods are well known in the art. For example, for applications requiring high selectivity, one will typically desire to employ relatively stringent conditions for the hybridization reactions, e.g., one will select relatively low salt and/or high temperature conditions, such as provided by about 0.02 M to about 0.10 M NaCl at temperatures of about 50° C. to about 70° C. Such high stringency conditions tolerate little, if any, mismatch between the probe and the template or target strand. It is generally appreciated that conditions can be rendered more stringent by the addition of increasing amounts of formamide. Other variations in hybridization reaction conditions are well known in the art (see for example, Sambrook et al., Molecular Cloning; A Laboratory Manual 2d ed. (1989)).

Hybridization conditions are based on the melting temperature (Tm) of the nucleic acid binding complex, as taught, e.g., in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of ordinary skill in the art, a maximum stringency hybridization can be used to identify or detect identical nucleotide sequences while an intermediate (or low) stringency hybridization can be used to identify or detect similar or related polynucleotide sequences.

In one aspect, the present invention employs nucleotide sequences that can hybridize to another nucleotide sequence under stringent conditions (e.g., 65° C. and 0.1×SSC {1×SSC=0.15 M NaCl, 0.015 M Na3 Citrate pH 7.0). Where the nucleotide sequence is double-stranded, both strands of the duplex, either individually or in combination, may be employed by the present invention. Where the nucleotide sequence is single-stranded, it is to be understood that the complementary sequence of that nucleotide sequence is also included within the scope of the present invention.

Stringency of hybridization refers to conditions under which polynuclcic acid hybrids are stable. Such conditions are evident to those of ordinary skill in the field. As known to those of ordinary skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridization reaction is performed under conditions of higher stringency, followed by washes of varying stringency.

As used herein, high stringency includes conditions that permit hybridization of only those nucleic acid sequences that form stable hybrids in 1 M Na+ at 65-68° C. High stringency conditions can be provided, for example, by hybridization in an aqueous solution containing 6×SSC, 5×Denhardt's, 1% SDS (sodium dodecyl sulphate), 0.1 Na+ pyrophosphate and 0.1 mg/ml denatured salmon sperm DNA as non-specific competitor. Following hybridization, high stringency washing may be done in several steps, with a final wash (about 30 minutes) at the hybridization temperature in 0.2-0.1×SSC, 0.1% SDS.

It is understood that these conditions may be adapted and duplicated using a variety of buffers, e.g., formamide-based buffers, and temperatures. Denhardt's solution and SSC are well known to those of ordinary skill in the art as are other suitable hybridization buffers (see, e.g., Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridization conditions are typically determined empirically, as the length and the GC content of the hybridizing pair also play a role.

Nucleic acid molecules that differ from the sequences of the primers and probes disclosed herein, are intended to be within the scope of the invention. Nucleic acid sequences that are complementary to these sequences, or that are hybridizable to the sequences described herein under conditions of standard or stringent hybridization, and also analogs and derivatives are also intended to be within the scope of the invention. Advantageously, such variations will differ from the sequences described herein by only a small number of nucleotides, for example by 1, 2, or 3 nucleotides.

Nucleic acid molecules corresponding to natural allelic variants, homologues (i.e., nucleic acids derived from other species), or other related sequences (e.g., paralogs) of the sequences described herein can be isolated based on their homology to the nucleic acids disclosed herein, for example by performing standard or stringent hybridization reactions using all or a portion of the known sequences as probes. Such methods for nucleic acid hybridization and cloning are well known in the art.

Similarly, a nucleic acid molecule detected in the methods of the invention may include only a fragment of the specific sequences described. Fragments provided herein are defined as sequences of at least 6 (contiguous) nucleic acids, a length sufficient to allow for specific hybridization of nucleic acid primers or probes, and are at most some portion less than a full-length sequence. Fragments may be derived from any contiguous portion of a nucleic acid sequence of choice. Derivatives and analogs may be full length or other than full length, if the derivative or analog contains a modified nucleic acid or amino acid, as described below.

Derivatives, analogs, homologues, and variants of the nucleic acids of the invention include, but are not limited to, molecules comprising regions that are substantially homologous to the nucleic acids of the invention, in various embodiments, by at least about 70%, 80%, 85%, 90%, 95%, 96%, 97%, 98%, or even 99% identity over a nucleic acid sequence of identical size or when compared to an aligned sequence in which the alignment is done by a computer homology program known in the art.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as (N_{ref}−N_{dif})*100/−N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N N_{ref}=8; N N_{dif}=2). “Homology” or “identity” can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, Proc Natl Acad Sci USA 1983; 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences. Without undue experimentation, the skilled artisan can consult with many other programs or references for determining percent homology.

In embodiments where expression of a particular gene is assessed by determining the expression of the protein product of the gene, any suitable assay for detecting protein levels and/or activity may be employed. For example, suitable protein activity assays include ubiquitination assays, kinase assays, protein-binding assays, DNA-binding and unwinding assays, and any other suitable assay for assessing the activity of the protein product of a translated gene according to the invention.

### Sampling

In order to determine the genotype or expression level of a particular SWI/SNF complex and/or MEDIATOR complex gene of a patient according to the methods of the present invention, it may be necessary to obtain a sample of genomic DNA or RNA from that patient. That sample of genomic DNA or RNA may be obtained from a sample of tissue or cells taken from that patient.

A sample may comprise any clinically relevant tissue sample, such as a tumor biopsy or fine needle aspirate, hair (including roots), skin, buccal swabs, saliva, or a sample of bodily fluid, such as blood, plasma, serum, lymph, ascitic fluid, cystic fluid, urine or nipple exudate. The sample may be taken from a human, or, in a veterinary context, from non-human animals such as ruminants, horses, swine or sheep, or from domestic companion animals such as felines and canines.

The tissue sample may be marked with an identifying number or other indicia that relates the sample to the individual patient from which the sample was taken. The identity of the sample advantageously remains constant throughout the methods of the invention thereby guaranteeing the integrity and continuity of the sample during extraction and analysis. Alternatively, the indicia may be changed in a regular fashion that ensures that the data, and any other associated data, can be related back to the patient from whom the data was obtained. The amount/size of sample required is known to those ordinarily skilled in the art.

Generally, the tissue sample may be placed in a container that is labeled using a numbering system bearing a code corresponding to the patient. Accordingly, the genotype of a particular patient is easily traceable.

In one embodiment of the invention, a sampling device and/or container may be supplied to the physician. The sampling device advantageously takes a consistent and reproducible sample from individual patients while simultaneously avoiding any cross-contamination of tissue. Accordingly, the size and volume of sample tissues derived from individual patients would be consistent.

According to the present invention, a sample of genomic DNA or RNA is obtained from the tissue sample of the patient of interest. Whatever source of cells or tissue is used, a sufficient amount of cells must be obtained to provide a sufficient amount of DNA or RNA for analysis. This amount will be known or readily determinable by those ordinarily skilled in the art.

DNA or RNA is isolated from the tissue/cells by techniques known to those ordinarily skilled in the art (see, e.g., U.S. Pat. Nos. 6,548,256 and 5,989,431, Hirota et al., Jinrui Idengaku Zasshi. September 1989; 34(3):217-23 and John et al., Nucleic Acids Res. Jan. 25, 1991; 19(2):408; the disclosures of which are incorporated by reference in their entireties). For example, high molecular weight DNA may be purified from cells or tissue using proteinase K extraction and ethanol precipitation. DNA may be extracted from a patient specimen using any other suitable methods known in the art.

In certain embodiments, target polynucleotide molecules are extracted from a sample taken from an individual afflicted with breast cancer. The sample may be collected in any clinically acceptable manner, but must be collected such that marker-derived polynucleotides (e.g., RNA) are preserved. mRNA or nucleic acids derived therefrom (e.g., cDNA or amplified DNA) are preferably labeled distinguishably from standard or control polynucleotide molecules, and both are simultaneously or independently hybridized to a microarray comprising one or more markers of resistance to anticancer treatment as described above. Alternatively, mRNA or nucleic acids derived therefrom may be labeled with the same label as the standard or control polynucleotide molecules, wherein the intensity of hybridization of each at a particular probe is compared.

Methods for preparing total and poly(A)+RNA are well known and are described generally in Sambrook et al., MOLECULAR CLONING—A LABORATORY MANUAL (2ND ED.), Vols. 1-3, Cold Spring Harbor Laboratory, Cold Spring Harbor, N.Y. (1989)) and Ausubel et al., CURRENT PROTOCOLS IN MOLECULAR BIOLOGY, vol. 2, Current Protocols Publishing, New York (1994)).

RNA may be isolated from eukaryotic cells by procedures that involve lysis of the cells and denaturation of the proteins contained therein. Cells of interest include wild-type cells (i.e., non-cancerous), drug-exposed wild-type cells, tumor- or tumor-derived cells, modified cells, normal or tumor cell line cells, and drug-exposed modified cells.

Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., Biochemistry 18:5294-5299 (1979)). Poly(A)+ RNA is selected by selection with oligo-dT cellulose (see Sambrook et al, MOLECULAR CLONING—A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol. If desired, RNase inhibitors may be added to the lysis buffer. Likewise, for certain cell types, it may be desirable to add a protein denaturation/digestion step to the protocol.

In certain embodiments, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or Sephadex™ (see Ausubel et al., CURRENT PROTOCOLS IN MOLECULAR BIOLOGY, vol. 2, Current Protocols Publishing, New York (1994). Once bound, poly(A)+mRNA is eluted from the affinity column using 2 mM EDTA/0.1% SDS. The sample of RNA can comprise a plurality of different mRNA molecules, each different mRNA molecule having a different nucleotide sequence. In a specific embodiment, the RNA sample is a mammalian RNA sample.

In a specific embodiment, total RNA or mRNA from cells are used in the methods of the invention. The source of the RNA can be cells of any animal, human, mammal, primate, non-human animal, dog, cat, mouse, rat, bird, yeast, eukaryote, etc. In specific embodiments, the method of the invention is used with a sample containing total mRNA or total RNA from 1×10⁶ cells or less. In another embodiment, proteins can be isolated from the foregoing sources, by methods known in the art, for use in expression analysis at the protein level.

In certain embodiments, expression of a biomarker according to the invention is measured using multiplex ligation-dependent probe amplification (MLPA) (see, e.g., WO 01/61033 and Schouten, J P et al. (2002) “Relative quantification of 40 nucleic acid sequences by multiplex ligation-dependent probe amplification” Nucleic Acids Res 30, e57) or reverse transcriptase MLPA (RT-MLPA) (see, e.g., Eldering, E et al. (2003) “Expression profiling via novel multiplex assay allows rapid assessment of gene regulation in defined signaling pathways” Nucleic Acids Res 31, e153). In RT-MLPA, mRNA is converted to eDNA by reverse transcriptase, followed by a normal MLPA reaction. In other embodiments, methylation-specific MLPA is employed to detect expression of a biomarker according to the instant invention (see, e.g., Nygren, A O et al. (2005) “Methylation-specific MLPA (MS-MPLA): simultaneous detection of CpG methylation and copy number changes of up to 40 sequences” Nucleic Acids Res 33, 14:e128).

### Arrays

As defined herein, a “nucleic acid array” refers to a plurality of unique nucleic acids (or “nucleic acid members”) attached to a support where each of the nucleic acid members is attached to a support in a unique pre-selected region.

In one embodiment, the nucleic acid member attached to the surface of the support is DNA. In another embodiment, the nucleic acid member attached to the surface of the support is either cDNA or oligonucleotides. In another embodiment, the nucleic acid member attached to the surface of the support is cDNA synthesized by polymerase chain reaction (PCR). In another embodiment, sequences bound to the array can be an isolated oligonucleotide, cDNA, EST or PCR product corresponding to any biomarker of the invention total cellular RNA is applied to the array.

Thus in one aspect, the present invention relates to an array comprising a nucleic acid which binds to at least one of the markers selected from the group consisting of an SWI/SNF complex and/or MEDIATOR complex gene for the determination of resistance to anticancer treatment, such as a RTK inhibitor compound (e.g., Herceptin).

Array technology and the various techniques and applications associated with it is described generally in numerous textbooks and documents. These include Lemieux et al., 1998, Molecular Breeding 4, 277-289, Schena and Davis. Parallel Analysis with Biological Chips. in PCR Methods Manual (eds. M. Innis, D. Gelfand, J. Sninsky), Schena and Davis, 1999, Genes, Genomes and Chips. In DNA Microarrays: A Practical Approach (ed. M. Schena), Oxford University Press, Oxford, UK, 1999), The Chipping Forecast (Nature Genetics special issue; January 1999 Supplement), Mark Schena (Ed.), Microarray Biochip Technology, (Eaton Publishing Company), Cortes, 2000, The Scientist 14[17]:25, Gwynne and Page, Microarray analysis: the next revolution in molecular biology, Science, 1999 August 6; and Eakins and Chu, 1999, Trends in Biotechnology, 17, 217-218.

Major applications for array technology include the identification of sequence (gene/gene mutation) and the determination of expression level (abundance) of genes. Gene expression profiling may make use of array technology, optionally in combination with proteomics techniques (Celis et al, 2000, FEBS Lett, 480(1):2-16; Lockhart and Winzeler, 2000, Nature 405(6788):827-836; Khan et al., 1999, 20(2):223-9). Other applications of array technology are also known in the art; for example, gene discovery, cancer research (Marx, 2000, Science 289: 1670-1672; Scherf, et al, 2000, Nat Genet; 24(3):236-44; Ross et al, 2000, Nat Genet. 2000 March; 24(3):227-35), SNP analysis (Wang et al, 1998, Science, 280(5366):1077-82), drug discovery, pharmacogenomics, disease diagnosis (for example, utilising microfluidics devices: Chemical & Engineering News, Feb. 22, 1999, 77(8):27-36), toxicology (Rockett and Dix (2000), Xenobiotica, 30(2):155-77; Afshari et al., 1999, Cancer Resl; 59(19):4759-60) and toxicogenomics (a hybrid of functional genomics and molecular toxicology).

In general, any library may be arranged in an orderly manner into an array, by spatially separating the members of the library. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others.

The samples (e.g., members of a library) are generally fixed or immobilized onto a solid phase, preferably a solid substrate, to limit diffusion and admixing of the samples. In particular, the libraries may be immobilized to a substantially planar solid phase, including membranes and non-porous substrates such as plastic and glass. Furthermore, the samples are preferably arranged in such a way that indexing (i.e., reference or access to a particular sample) is facilitated. Typically the samples are applied as spots in a grid formation. Common assay systems may be adapted for this purpose. For example, an array may be immobilized on the surface of a microplate, either with multiple samples in a well, or with a single sample in each well. Furthermore, the solid substrate may be a membrane, such as a nitrocellulose or nylon membrane (for example, membranes used in blotting experiments). Alternative substrates include glass, or silica-based substrates. Thus, the samples are immobilized by any suitable method known in the art, for example, by charge interactions, or by chemical coupling to the walls or bottom of the wells, or the surface of the membrane. Other means of arranging and fixing may be used, for example, pipetting, drop-touch, piezoelectric means, ink-jet and bubblejet technology, electrostatic application, etc. In the case of silicon-based chips, photolithography may be utilized to arrange and fix the samples on the chip.

The samples may be arranged by being “spotted” onto the solid substrate; this may be done by hand or by making use of robotics to deposit the sample. In general, arrays may be described as macroarrays or microarrays, the difference being the size of the sample spots. Macroarrays typically contain sample spot sizes of about 300 microns or larger and may be easily imaged by existing gel and blot scanners. The sample spot sizes in microarrays are typically less than 200 microns in diameter and these arrays usually contain thousands of spots. Thus, microarrays may require specialized robotics and imaging equipment, which may need to be custom made. Instrumentation is described generally in a review by Cortese, 2000, The Scientist 14[11]:26.

Techniques for producing immobilized libraries of DNA molecules have been described in the art. Generally, most prior art methods described how to synthesize single-stranded nucleic acid molecule libraries, using for example masking techniques to build up various permutations of sequences at the various discrete positions on the solid substrate. U.S. Pat. No. 5,837,832 describes an improved method for producing DNA arrays immobilized to silicon substrates based on very large scale integration technology. In particular, U.S. Pat. No. 5,837,832 describes a strategy called “tiling” to synthesize specific sets of probes at spatially-defined locations on a substrate which may be used to produced the immobilized DNA libraries of the present invention. U.S. Pat. No. 5,837,832 also provides references for earlier techniques that may also be used. Arrays may also be built using photo deposition chemistry.

To aid detection, labels are typically used—such as any readily detectable reporter, for example, a fluorescent, bioluminescent, phosphorescent, radioactive, etc. reporter. Labelling of probes and targets is also disclosed in Shalon et al., 1996, Genome Res 6(7):639-45.

Examples of DNA arrays include where probe eDNA (500-5,000 bases long) is immobilized to a solid surface such as glass using robot spotting and exposed to a set of targets either separately or in a mixture. This method is widely considered as having been developed at Stanford University (Ekins and Chu, 1999, Trends in Biotechnology, 1999, 17, 217-218).

Another example of a DNA array is where an array of oligonucleotides (20-25-mer oligos, preferably, 40-60 mer oligos) or peptide nucleic acid (PNA) probes are synthesized either in situ (on-chip) or by conventional synthesis followed by on-chip immobilization. The array is exposed to labelled sample DNA, hybridized, and the identity/abundance of complementary sequences are determined. Such a DNA chip is sold by Affymetrix, Inc., under the GeneChip® trademark. Agilent and Nimblegen also provide suitable arrays (eg. genomic tiling arrays).

In other embodiments, high throughput DNA sequencing promises to become an affordable and more quantitative alternative for microarrays to analyze large collections of DNA sequences. Examples of high-throughput sequencing approaches are listed in E. Y. Chan, Mutation Research 573 (2005) 13-40 and include, but are not limited to, near-term sequencing approaches such as cycle-extension approaches, polymerase reading approaches and exonuclease sequencing, revolutionary sequencing approaches such as DNA scanning and nanopore sequencing and direct linear analysis. Examples of current high-throughput sequencing methods are 454 (pyro)sequencing, Solexa Genome Analysis System, Agencourt SOLiD sequencing method (Applied Biosystems), MS-PET sequencing (Ng et al., 2006, http://nar.oxfordjournals.org/cgi/content/full/34/12/e84).

### Probes

As used herein, the term “probe” refers to a molecule (e.g., an oligonucleotide, whether occurring naturally as in a purified restriction digest or produced synthetically, recombinantly or by PCR amplification), that is capable of hybridizing to another molecule of interest (e.g., another oligonucleotide). When probes are oligonucleotides they may be single-stranded or double-stranded. Probes are useful in the detection, identification and isolation of particular targets (e.g., gene sequences). As described herein, it is contemplated that probes used in the present invention may be labelled with a label so that is detectable in any detection system, including, but not limited to enzyme (e.g., ELISA, as well as enzyme-based histochemical assays), fluorescent, radioactive, and luminescent systems.

With respect to arrays and microarrays, the term “probe” is used to refer to any hybridizable material that is affixed to the array for the purpose of detecting a nucleotide sequence that has hybridized to said probe. Preferably, these probes are 25-60 mers or longer.

The present invention further encompasses probes according to the present invention that are immobilized on a solid or flexible support, such as paper, nylon or other type of membrane, filter, chip, glass slide, microchips, microbeads, or any other such matrix, all of which are within the scope of this invention.

The primers and probes described herein may be readily prepared by, for example, directly synthesizing the fragment by chemical means or by introducing selected sequences into recombinant vectors for recombinant production. Methods for making a vector or recombinants or plasmid for amplification of the fragment either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6,312,683; U.S. patent application Ser. No. 920,197, filed Oct. 16, 1986; WO 90/01543; WO91/11525; WO 94/16716; WO 96/39491; WO 98/33510; EP 265785; EP 0 370 573; Andreansky et al., Proc. Natl. Acad. Sci. USA 1996; 93:11313-11318; Ballay et al., EMBO J. 1993; 4:3861-65; Felgner et al., J. Biol. Chem. 1994; 269:2550-2561; Frolov et al., Proc. Natl. Acad. Sci. USA 1996; 93:11371-11377; Graham, Tibtech 1990; 8:85-87; Grunhaus et al., Sem. Virol. 1992; 3:237-52; Ju et al., Diabetologia 1998; 41:736-739; Kitson et al., J. Virol. 1991; 65:3068-3075; McClements et al., Proc. Natl. Acad. Sci. USA 1996; 93:11414-11420; Moss, Proc. Natl. Acad. Sci. USA 1996; 93:11341-11348; Paoletti, Proc. Natl. Acad. Sci. USA 1996; 93:11349-11353; Pennock et al., Mol. Cell. Biol. 1984; 4:399-406; Richardson (Ed), Methods in Molecular Biology 1995; 39, “Baculovirus Expression Protocols,” Humana Press Inc.; Smith et al. (1983) Mol. Cell. Biol. 1983; 3:2156-2165; Robertson et al., Proc. Natl. Acad. Sci. USA 1996; 93:11334-11340; Robinson et al., Sem. Immunol. 1997; 9:271; and Roizman, Proc. Natl. Acad. Sci. USA 1996; 93:11307-11312. Strategies for probe design are described in WO95/11995, EP 717,113 and WO97/29212.

In order to generate data from array-based assays a signal is detected that signifies the presence of or absence of hybridization between a probe and a nucleotide sequence. The present invention further contemplates direct and indirect labelling techniques. For example, direct labelling incorporates fluorescent dyes directly into the nucleotide sequences that hybridize to the array-associated probes (e.g., dyes are incorporated into nucleotide sequence by enzymatic synthesis in the presence of labelled nucleotides or PCR primers). Direct labelling schemes yield strong hybridization signals, typically using families of fluorescent dyes with similar chemical structures and characteristics, and are simple to implement. In some embodiments comprising direct labelling of nucleic acids, cyanine or alexa analogs are utilized in multiple-fluor comparative array analyses. In other embodiments, indirect labelling schemes can be utilized to incorporate epitopes into the nucleic acids either prior to or after hybridization to the microarray probes. One or more staining procedures and reagents are used to label the hybridized complex (e.g., a fluorescent molecule that binds to the epitopes, thereby providing a fluorescent signal by virtue of the conjugation of dye molecule to the epitope of the hybridised species).

Oligonucleotide sequences used as probes according to the present invention may be labeled with a detectable moiety. Various labeling moieties are known in the art. Said moiety may be, for example, a radiolabel (e.g., 3H, 1251, 35S, 14C, 32P, etc.), detectable enzyme (e.g. horse radish peroxidase (HRP), alkaline phosphatase etc.), a fluorescent dye (e.g., fluorescein isothiocyanate, Texas red, rhodamine, Cy3, Cy5, Bodipy, Bodipy Far Red, Lucifer Yellow, Bodipy 630/650-X, Bodipy R60-X and 5-CR 6G, and the like), a colorimetric label such as colloidal gold or colored glass or plastic (e.g. polystyrene, polypropylene, latex, etc.), beads, or any other moiety capable of generating a detectable signal such as a colorimetric, fluorescent, chemiluminescent or electrochemiluminescent (ECL) signal.

Probes may be labeled directly or indirectly with a detectable moiety, or synthesized to incorporate the detectable moiety. In one embodiment, a detectable label is incorporated into a nucleic acid during at least one cycle of a cyclic polymerase-mediated amplification reaction. For example, polymerases can be used to incorporate fluorescent nucleotides during the course of polymerase-mediated amplification reactions. Alternatively, fluorescent nucleotides may be incorporated during synthesis of nucleic acid primers or probes. To label an oligonucleotide with the fluorescent dye, one of conventionally-known labeling methods can be used (Nature Biotechnology, 14, 303-308, 1996; Applied and Environmental Microbiology, 63, 1143-1147, 1997; Nucleic Acids Research, 24, 4532-4535, 1996). An advantageous probe is one labeled with a fluorescent dye at the 3′ or 5′ end and containing G or C as the base at the labeled end. If the 5′ end is labeled and the 3′ end is not labeled, the OH group on the C atom at the 3′-position of the 3′ end ribose or deoxyribose may be modified with a phosphate group or the like although no limitation is imposed in this respect.

Spectroscopic, photochemical, biochemical, immunochemical, electrical, optical or chemical means can be used to detect such labels. The detection device and method may include, but is not limited to, optical imaging, electronic imaging, imaging with a CCD camera, integrated optical imaging, and mass spectrometry. Further, the amount of labeled or unlabeled probe bound to the target may be quantified. Such quantification may include statistical analysis. In other embodiments the detection may be via conductivity differences between concordant and discordant sites, by quenching, by fluorescence perturbation analysis, or by electron transport between donor and acceptor molecules.

In yet another embodiment, detection may be via energy transfer between molecules in the hybridization complexes in PCR or hybridization reactions, such as by fluorescence energy transfer (FET) or fluorescence resonance energy transfer (FRET). In FET and FRET methods, one or more nucleic acid probes are labeled with fluorescent molecules, one of which is able to act as an energy donor and the other of which is an energy acceptor molecule. These are sometimes known as a reporter molecule and a quencher molecule respectively. The donor molecule is excited with a specific wavelength of light for which it will normally exhibit a fluorescence emission wavelength. The acceptor molecule is also excited at this wavelength such that it can accept the emission energy of the donor molecule by a variety of distance-dependent energy transfer mechanisms. Generally the acceptor molecule accepts the emission energy of the donor molecule when they are in close proximity (e.g., on the same, or a neighboring molecule). FET and FRET techniques are well known in the art. See for example U.S. Pat. Nos. 5,668,648, 5,707,804, 5,728,528, 5,853,992, and 5,869,255 (for a description of FRET dyes), Tyagi et al. Nature Biotech. vol. 14, p 303-8 (1996), and Tyagi et al., Nature Biotech. vol 16, p 49-53 (1998) (for a description of molecular beacons for FET), and Mergny et al. Nucleic Acid Res. vol 22, p 920-928, (1994) and Wolf et al. PNAS vol 85, p 8790-94 (1988) (for general descriptions and methods fir FET and FRET), each of which is hereby incorporated by reference.

The probes for use in an array of the invention may be greater than 40 nucleotides in length and may be isothermal.

In some embodiments, the probes, array of probes or set of probes will be immobilized on a support. Supports (e.g., solid supports) can be made of a variety of materials, such as glass, silica, plastic, nylon or nitrocellulose. Supports are preferably rigid and have a planar surface. Supports typically have from about 1-10,000,000 discrete spatially addressable regions, or cells. Supports having about 10-1,000,000 or about 100-100,000 or about 1000-100,000 cells are common. The density of cells is typically at least about 1000, 10,000, 100,000 or 1,000,000 cells within a square centimeter. In some supports, all cells are occupied by pooled mixtures of probes or a set of probes. In other supports, some cells are occupied by pooled mixtures of probes or a set of probes, and other cells are occupied, at least to the degree of purity obtainable by synthesis methods, by a single type of oligonucleotide.

Arrays of probes or sets of probes may be synthesized in a step-by-step manner on a support or can be attached in presynthesized form. One method of synthesis is VLSIPS™ (as described in U.S. Pat. No. 5,143,854 and EP 476,014), which entails the use of light to direct the synthesis of oligonucleotide probes in high-density, miniaturized arrays. Algorithms for design of masks to reduce the number of synthesis cycles are described in U.S. Pat. No. 5,571,639 and U.S. Pat. No. 5,593,839. Arrays can also be synthesized in a combinatorial fashion by delivering monomers to cells of a support by mechanically constrained flowpaths, as described in EP 624,059. Arrays can also be synthesized by spotting reagents on to a support using an ink jet printer (see, for example, EP 728,520).

### Data Analysis

Data analysis is also an important part of an experiment involving arrays. The raw data from an array experiment typically are images, which need to be transformed into matrices—tables where rows represent, for example, genes, columns represent, for example, various samples such as tissues or experimental conditions, and numbers in each cell for example characterize the expression of a particular sequence (for example, a second sequence that has ligated to the first (target) nucleotide sequence) in the particular sample. These matrices have to be analyzed further, if any knowledge about the underlying biological processes is to be extracted. Methods of data analysis (including supervised and unsupervised data analysis as well as bioinformatics approaches) are disclosed in Brazma and Vilo J (2000) FEBS Lett 480(1):17-24.

### Kits

The materials for use in the methods of the present invention are ideally suited for preparation of kits. Oligonucleotides may be provided in containers that can be in any form, e.g., lyophilized, or in solution (e.g., a distilled water or buffered solution), etc. In one aspect of the present invention, there is provided a kit comprising a set of probes as described herein, an array and optionally one or more labels. In another aspect, there is provided an RT-MLPA kit comprising a set of reverse transcriptase primers as described herein, and appropriate ligases, buffers, and PCR primers. In the kits of the invention, a set of instructions will also typically be included.

The oligonucleotide primers and probes of the present invention have commercial applications in prognostic kits for the detection of the expression level of a gene, such as a MEDIATOR complex and/or SWI/SNF complex gene in the tumor cells of a patient. A test kit according to the invention may comprise any of the oligonucleotide primers or probes according to the invention. Such a test kit may additionally comprise one or more reagents for use in cyclic polymerase mediated amplification reactions, such as DNA polymerases, nucleotides (dNTPs), buffers, and the like. A kit according to the invention may also include, for example, a lysing buffer for lysing cells contained in the specimen.

A test kit according to the invention may comprise a pair of oligonucleotide primers according to the invention and a probe comprising an oligonucleotide according to the invention. Advantageously, the kit further comprises additional means, such as reagents, for detecting or measuring the binding of the primers and probes of the present invention, and also ideally a positive and negative control.

The invention will now be further described by way of the following non-limiting examples.

### Example 1

Identification of MED12, ARID1A and SMARCE1 as Molecular Determinants of Resistance to ALK Inhibitors in an EML4-ALK Positive NSCLC Cell Line Using a shRNA Barcode Screen

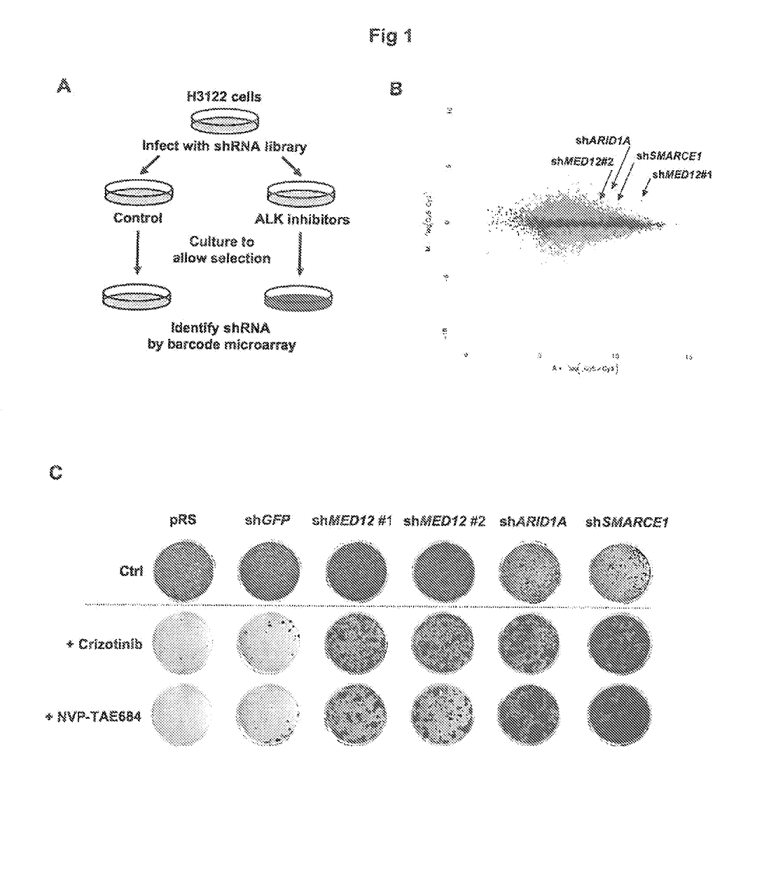

The ALK inhibitors crizotinib and NVP-TAE684 potently inhibit the human NSCLC cell lines that harbor EML4-ALK translocations (Galkin et al., 2007; Koivunen et al., 2008; Soda et al., 2007). The NSCLC cell line H3122 carries the EML4-ALK translocation and is exquisitely sensitive to ALK inhibitors. To identify novel determinants of resistance to ALK inhibitors in NSCLC cell lines, Applicants performed a large-scale RNAi-based loss-of-function genetic screen using a collection of 24,000 short hairpin (shRNA) vectors targeting 8,000 human genes (Berns et al., 2004; Brummelkamp et al., 2002). Applicants used a barcoding technology to identify genes whose suppression causes resistance to ALK inhibitors (Brummelkamp et al., 2006; Holzel et al.). The entire shRNA library was introduced into H3122 cells by retroviral infection and cells were plated at low density with or without ALK inhibitors (FIG. 1A). After four weeks of incubation with ALK inhibitors and the emergence of resistant cell clones, genomic DNA was isolated from treated and untreated cultures. The stably integrated shRNA cassettes (19-mer bar code sequences) were recovered by PCR from genomic DNA. The relative abundance of individual shRNA vectors was quantified by hybridization of the PCR products to microarrays harboring all 24,000 barcode sequences. The barcode screen was carried out in triplicate and the combined results are shown in FIG. 1B. Each dot in the M/A-plot represents one individual shRNA vector in the library. M- and A-values reflect relative enrichment and hybridization signal intensity. Reproducible outliers are generally located in the right upper corner. Low-intensity spots are prone to technical artifacts and thus unreliable. Therefore Applicants restricted their candidate selection by applying M/A cut-off values of M>7.5 and A>7.5 as previously described (Holzel et al.). The identification of independent shRNAs against the same gene or single shRNAs targeting multiple components of the same complex or signaling pathway strongly suggest a genuine hit from the screen. Applying these filter criteria, Applicants identified shRNAs against the genes MED12, ARID1A and SMARCE1.

### MED12, ARID1A and SMARCE1 are Components of Large Multi-Subunit Mediator and SWI/SNF Complexes Involved in Transcriptional Regulation and Chromatin Remodeling

The MEDI 2 gene encodes for a component of the large mediator complex (˜2 MDa) that contains at least 33 different subunits and associates with RNA polymerase II at the promoters of genes (Malik and Roeder). Thereby, the Mediator complex is involved in transcriptional regulation. Initially it was thought that the mediator complex is exclusively required for active transcription of genes, but recent studies suggest additional and broader roles in transcriptional regulation, such as epigenetic silencing. In particular, MED12 was implicated in contributing to silencing of neuronal genes in non-neuronal cells by the recruitment of the H3K9 histone methyltransferase EHMT2 (G9a) in a REST dependent manner (Ding et al., 2008). Interestingly, mutations in MED12 are causal for some rare mental retardation syndromes and aberrant gene regulation might contribute to the phenotypic manifestations of these diseases (Risheg et al., 2007; Schwartz et al., 2007). In general, only a few studies have addressed the specific function of individual components of the mediator complex.

ARID1A and SMARCE1 are both components of the SWI/SNF chromatin-remodeling complex (Reisman et al., 2009). The SWI/SNF complex is also a large multi-subunit complex that contains two mutual exclusive but non-redundant subunits with ATPase activity. The ATPases SMARCA2 (BRM1) and SMARCA4 (BRG1) are required for the ATP dependent re-positioning of histones within the chromatin. This ATP-dependent chromatin remodeling activity impacts diverse chromatin related biological processes such as gene transcription and DNA repair. The SWI/SNF complex is conserved throughout evolution from yeast to man. Hence, it is remarkable that several subunits of the SWI/SNF complex have been identified as tumor suppressors. Deletions of SMARCB1 (INI1, BAF47) are found in malignant rhabdoid tumors, a highly aggressive childhood cancer (Versteege et al., 1998). Inactivating truncating mutations of ARID1A and PBRMI were found in more than 50% and 40% of clear cell ovarian and renal cancer, respectively (Jones et al.; Varela et al.). SMARCA4 (BRG1) is frequently mutated in NSCLC cell lines, but also in primary tumors (Medina et al., 2008; Rodriguez-Nieto et al.). In conclusion, there is substantial evidence in the literature that specific components of the SWI/SNF complex function as tumor suppressors in a tumor type dependent manner, but the molecular basis of this selectivity remains unknown.

Validation of shRNA Barcode Screen Results

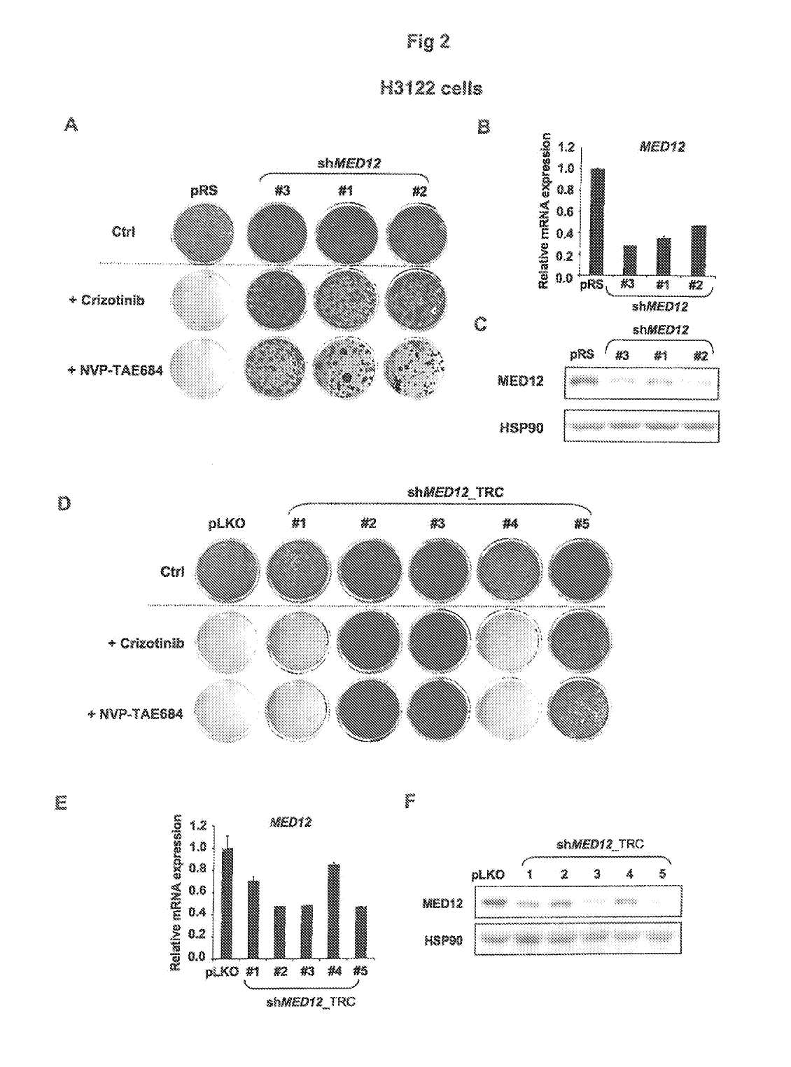

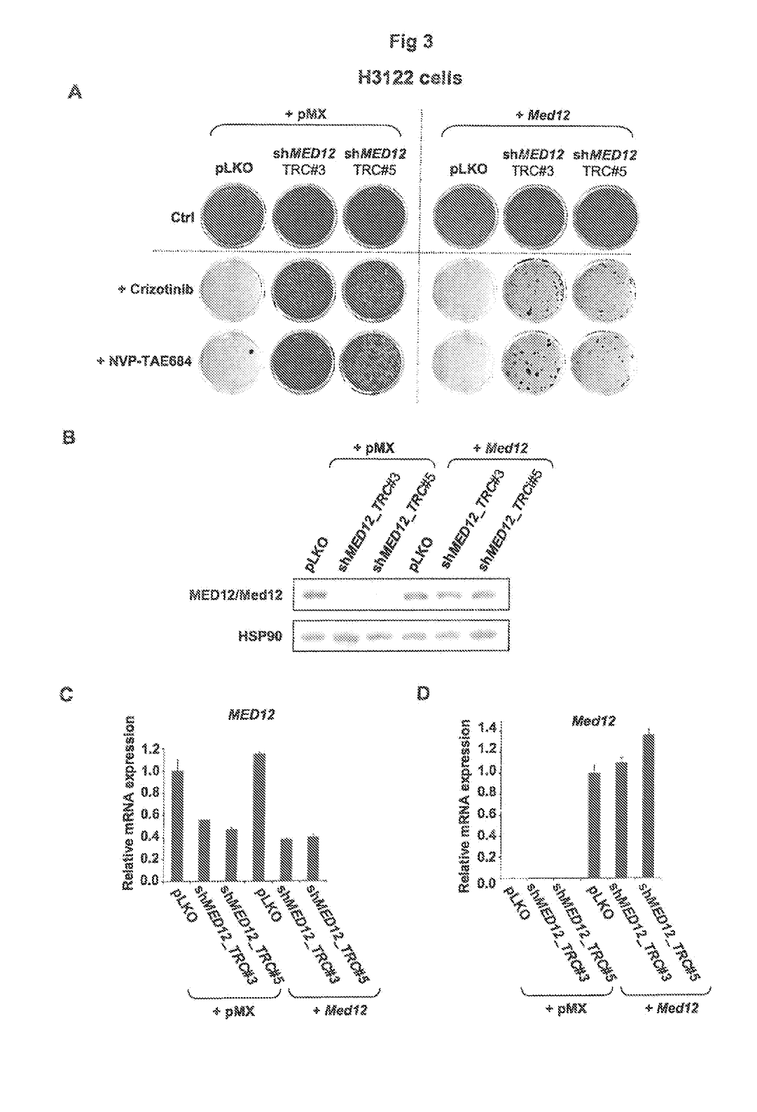

To validate the results of their screen, Applicants individually introduced the respective knockdown vectors from the NKI shRNA library against MED12 (#1 and #2), ARID1A and SMARCE1 into H3122 cells by retroviral infections and confirmed that all four shRNA vectors confer resistance to the ALK inhibitors crizotinib and NVP-TAE684 in H3122 cells (FIG. 1C). To rule out ‘off-target’ effects, a common problem in the field of RNAi screening, Applicants only consider a gene identified from the screen as a genuine hit, if at least two independent shRNAs suppress the expression of the target mRNA and also confer resistance to the ALK inhibitors (Echeverri et al., 2006). Even though Applicants identified already two independent shRNAs targeting MED12 from the screen, Applicants generated a third non-overlapping retroviral shRNA vector against MED12 (#3) that recapitulated the resistance to ALK inhibitors (FIG. 2A). All three shMED12 knockdown vectors potently suppressed MED12 mRNA and protein levels as determined by qRT-PCR and immunoblotting (FIGS. 2B and 2C). Furthermore, Applicants retrieved five independent lentiviral shRNA vectors against MED12 from the human TRC shRNA collection and infected H3122 cells. Suppression of MED12 mRNA level was confirmed by qRT-PCR and immunoblotting (FIGS. 2E and 2F). Indeed, the three best shMED12_TRC knockdown vectors (shMED12_TRC#2, #3, #5) conferred resistance to the ALK inhibitors (FIG. 2D). In conclusion, Applicants demonstrated that multiple independent non-overlapping shRNAs against MED12 cause resistance to ALK inhibitors strongly suggesting that MED12 is a genuine on-target hit from the screen. As a further proof, Applicants introduced the shRNA resistant mouse MED12 cDNA into H3122 cells expressing human MED12 specific shRNAs (shMED12_TRC#3 and TRC#5). As a control, Applicants infected MED12 knockdown cells with the pMX empty vector. Applicants confirmed reconstitution of MED12/Med12 protein levels at physiological levels in MED12 knockdown cells using a MED12 specific antibody that recognizes MED12/Med12 from both species (FIG. 3B). Importantly, Med12 reconstitution restored sensitivity to ALK inhibitors in MED12 knockdown cells (FIG. 3A). Applicants also verified a persistent knockdown of human MED12 mRNA in cells expressing the mouse Med12 cDNA by qRT-PCR using a human MED12 specific primer pair (FIG. 3C). In turn, Applicants also confirmed expression of the mouse Med12 cDNA using a mouse Med12 specific primer pair (FIG. 3D). In summary, these experiments demonstrate that MED12 is a genuine on-target hit from the ALK inhibitor shRNA resistance screen.

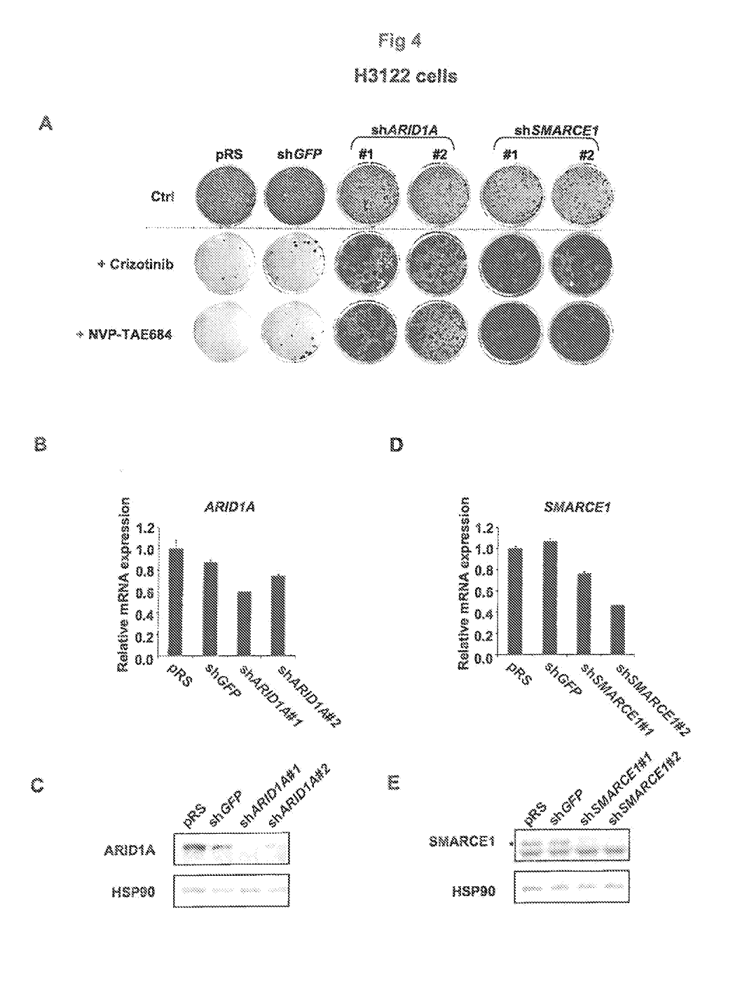

Next, Applicants validated that ARID1A and SMARCE1 are on-target hits causally involved in the resistance to ALK inhibitors. As Applicants have only identified single shRNAs (shARID1A#1, shSMARCE1#1) against these genes from the barcode screen, Applicants generated additional non-overlapping shRNAs against ARID1A and SMARCE1 (shARID1A#2, shSMARCE1#2) and introduced them into H3122 cells by retroviral infection. The independent shRNAs recapitulated the resistance to ALK inhibitors (FIG. 4A). It is noteworthy that knockdown of either ARID1A or SMARCE1 impaired proliferation of H3122 cells in the absence of the inhibitors. Applicants confirmed the suppression of ARID1A and SMARCE1 mRNA und protein levels by qRT-PCR and immunoblotting (FIG. 4B-4E). Again, these results show that ARID1A and SMARCE1 are genuine on-target hits from the screen.

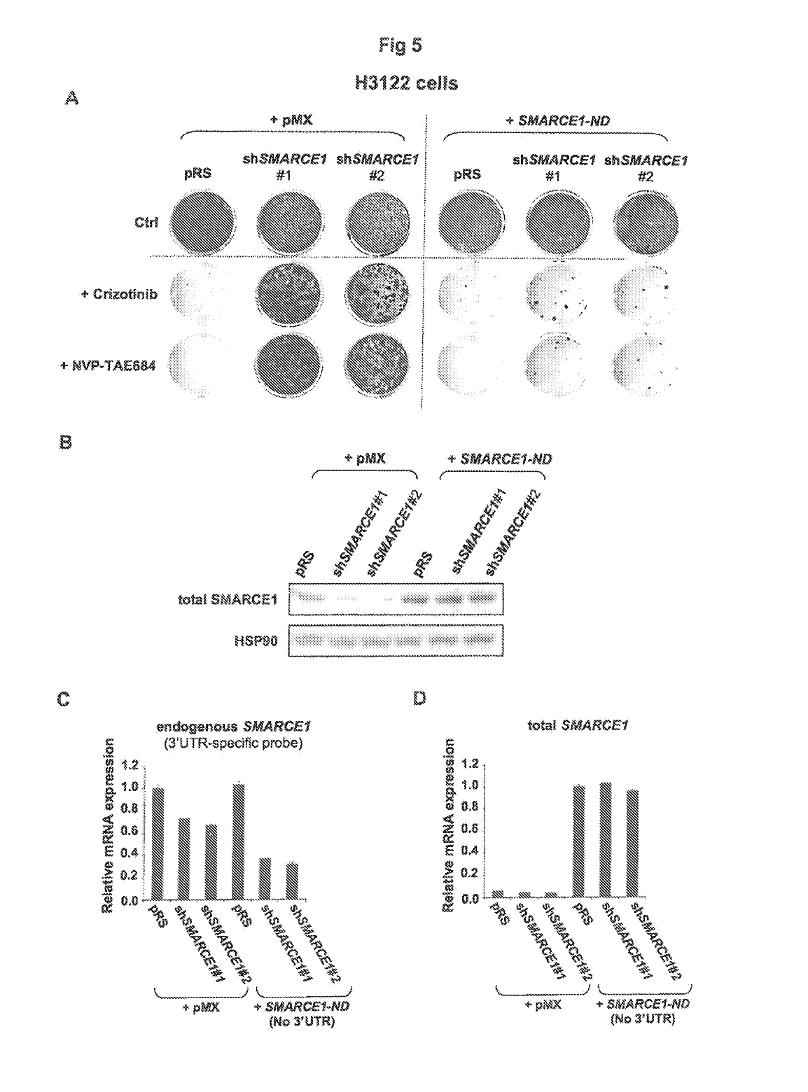

Next, Applicants introduced silent mutations into a human SMARCE1 cDNA expression construct and thereby generated two separate shRNA resistant (non-degradable, NI)) forms of SMARCE1 (SMARCE1-ND) that cannot be targeted by shSMARCE1#1 and shSMARCE #2. H3122 cells stably infected with pRS, shSMARCE1#1 or #2 were super-infected with retroviral expression constructs encoding for the respective non-degradable forms of SMARCE1 or the pMx empty control vector. Reconstitution of SMARCE1 restored sensitivity of SMARCE1 knockdown cells to ALK inhibitors (FIG. 5A). Applicants confirmed reconstituted SMARCE1 protein levels in SMARCE1 knockdown cells by immunoblotting using an SMARCE1 specific antibody, again achieving close to endogenous level of SMARCE1 (FIG. 5B). Applicants also verified a persistent knockdown of the endogenous human SMARCE1 mRNA in cells expressing the non-degradable SMARCE1 cDNAs by qRT-PCR using a human SMARCE1 3′UTR specific primer pair (FIG. 5C). In turn, Applicants also confirmed expression of the SMARCE1 cDNA using an open reading frame specific primer pair detecting endogenous and ectopic (total) SMARCE1 (FIG. 5D). In summary, these experiments demonstrate that SMARCE1 is a genuine on-target hit from the ALK inhibitor shRNA resistance screen.

### MED12, ARID1A and SMARCE1 are Molecular Determinants of Resistance to Tyrosine Kinase Inhibitors in Multiple NSCLC Cell Lines

Next, Applicants addressed the context dependency of their findings by studying independent NSCLC cell lines. The RAS/PI3K signaling cascade is a common denominator of all activated tyrosine kinases in NSCLC such as the EGFR (Pao and Chmielecki). Therefore, Applicants hypothesized that loss of MED12, SMARCE1 and ARID1A might also confer resistance to other tyrosine kinase inhibitors in cell lines that harbor respective activating mutations or amplifications.

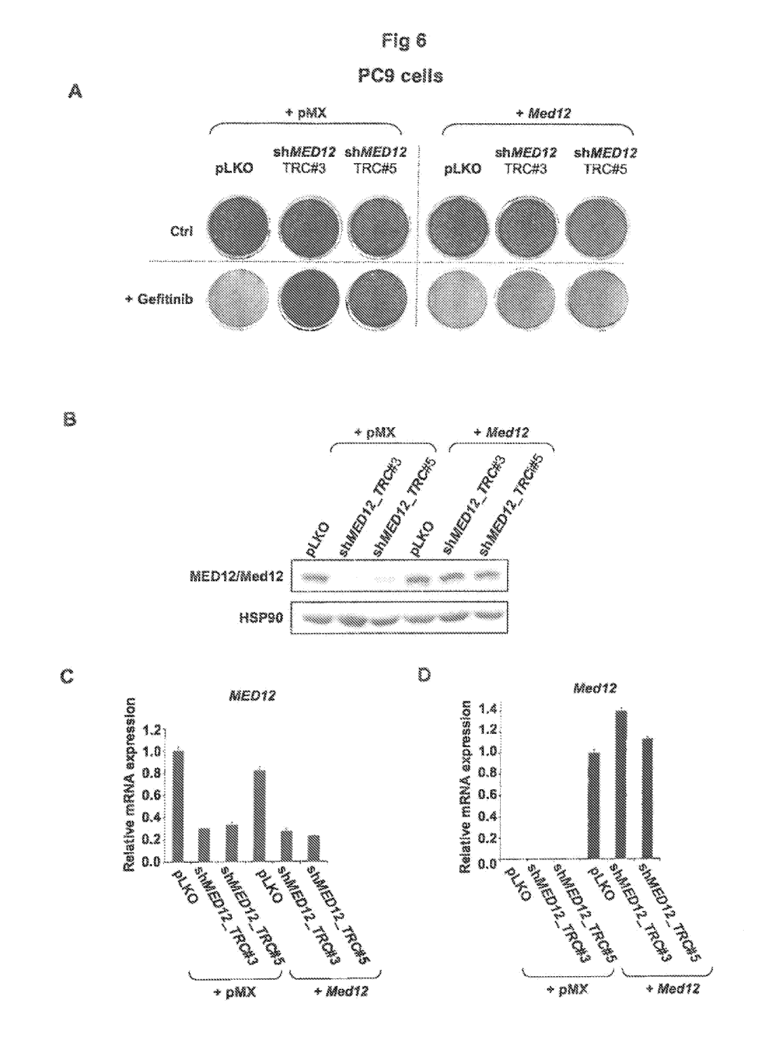

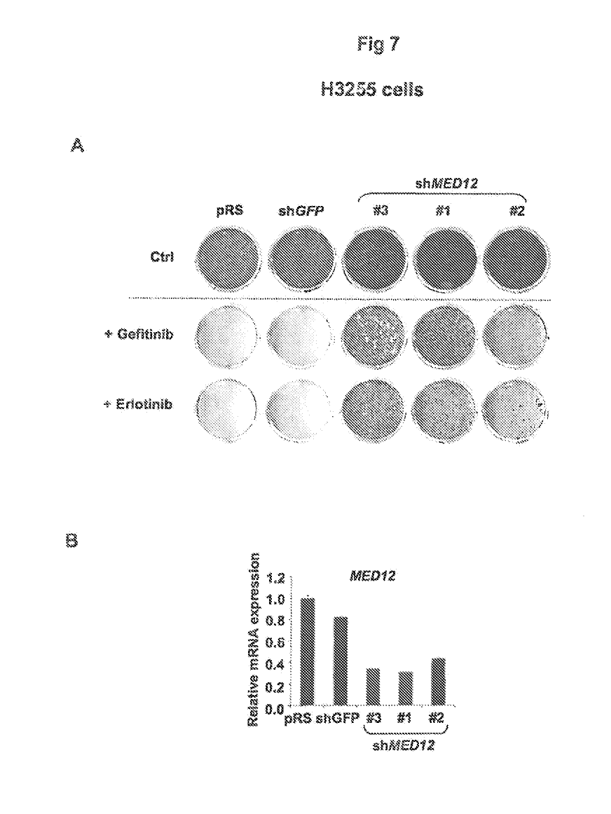

NSCLC with activating mutations of the EGFR can be effectively treated with the EGFR inhibitors gefitinib and erlotinib. Several NSCLC cell lines with EGFR mutations (PC9, H3255) were identified that are exquisitely sensitive to gefitinib and erlotinib at low nanomolar concentrations. Applicants introduced MED12 specific shRNAs (shMED12_TRC#3 and #5) into PC9 cells (EGFRdelE746-A750). Suppression of MED12 rendered PC9 cells insensitive to the EGFR inhibitor gefitinib (FIG. 6A, left panel). In addition, reconstitution of PC9 MED12-knockdown cells with the mouse Med12 cDNA restored their sensitivity to gefitinib (FIG. 6A, right panel). Using an antibody that recognizes human and mouse MED12/Med12, Applicants confirmed the suppression and restoration of MED12 protein level in the indicated PC9 cell lines by immunoblotting (FIG. 6B). Applicants also verified persistent knockdown of endogenous MED12 by qRT-PCR using a human MED12 specific primer pair (FIG. 6C). Likewise, Applicants controlled the ectopic expression of the mouse Med12 cDNA by qRT-PCR using a mouse Med12 specific primer pair (FIG. 6D). Furthermore, H3255 (EGFRL858R) cells were stably infected with three MED12 shRNA or control constructs (pRS and shGFP) and incubated with two EGFR inhibitors (gefitinib and erlotinib). Control cells were effectively eradicated, whereas shMED12 cells were insensitive to the treatment with the inhibitors (FIG. 7A). Applicants confirmed suppression of MED12 by qRT-PCR (FIG. 7B). In conclusion, Applicants demonstrated that loss of MED12 confers resistance to ALK and EGFR tyrosine kinase inhibitors in multiple NSCLC cell lines.

Next, Applicants asked whether ARID1A determines sensitivity to tyrosine kinase inhibitors in multiple NSCLC cell lines (context dependency). Applicants introduced the retroviral shRNA vectors against ARID1A (#1 and #2) or control vectors (pRS and shGFP) into PC9 (EGFRdelE746-A750) and H1993 (MET-amplified) cells (FIGS. 1A and 1C). Suppression of ARID1A conferred resistance to the EGFR inhibitor gefitinib and the MET inhibitor crizotinib in PC9 and H111993 cells, respectively. Knockdown of ARID1A mRNA was confirmed by qRT-PCR (FIGS. 2B and 2D).

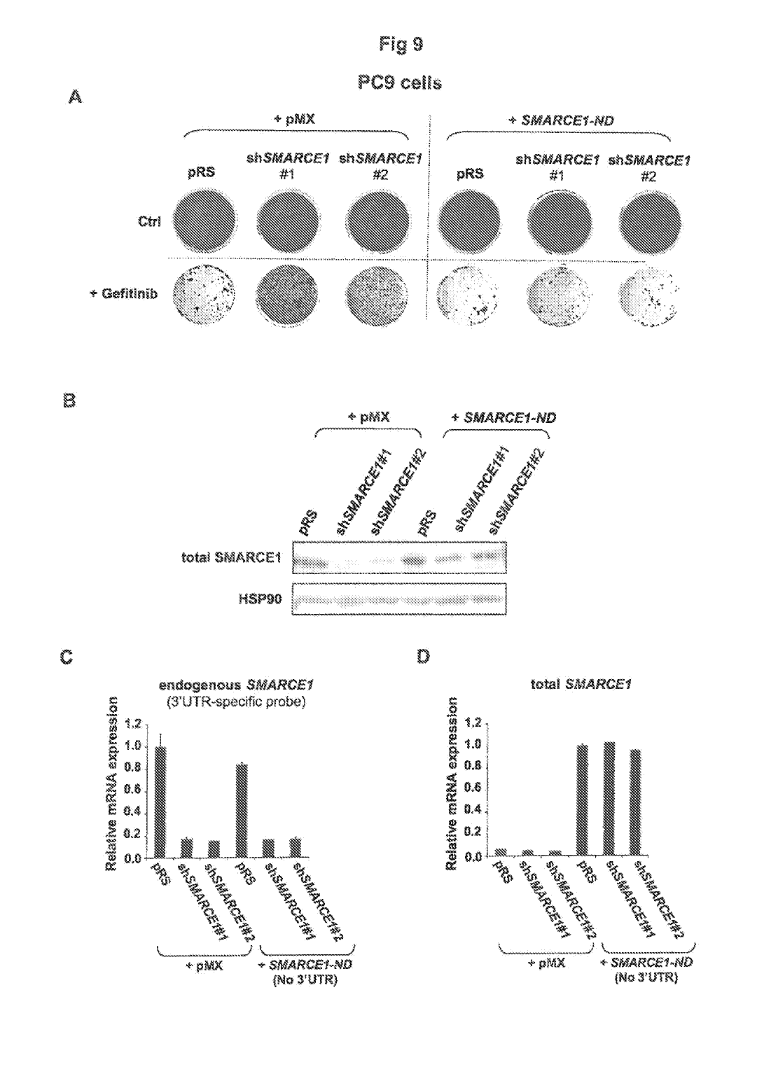

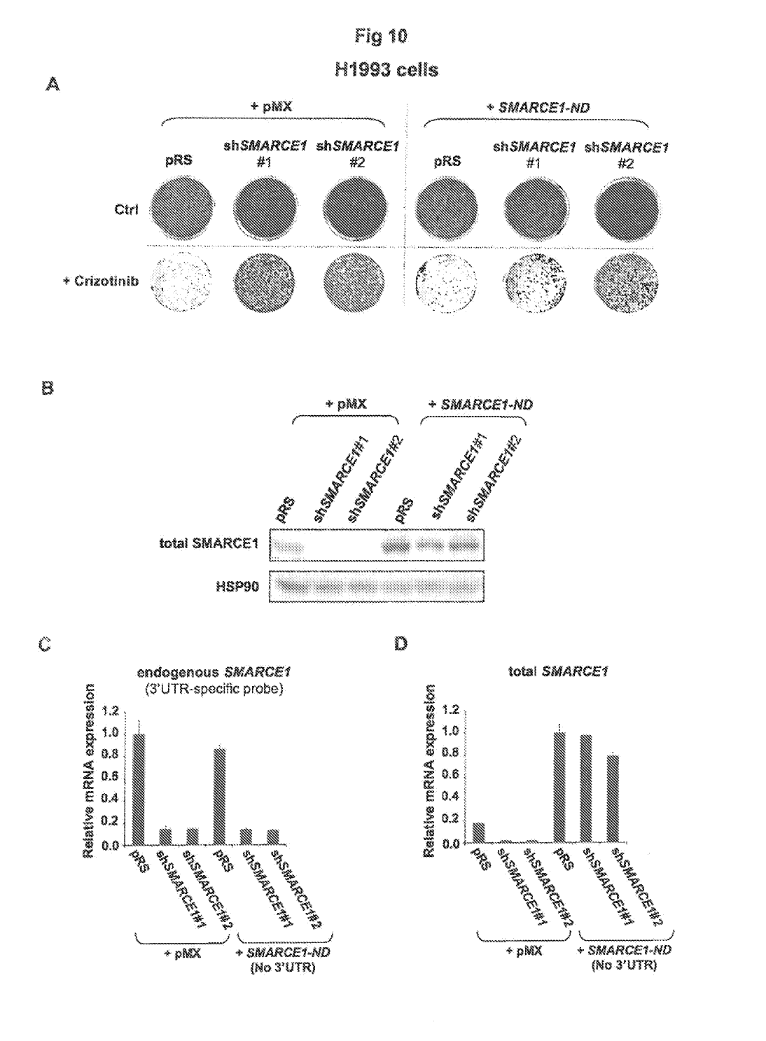

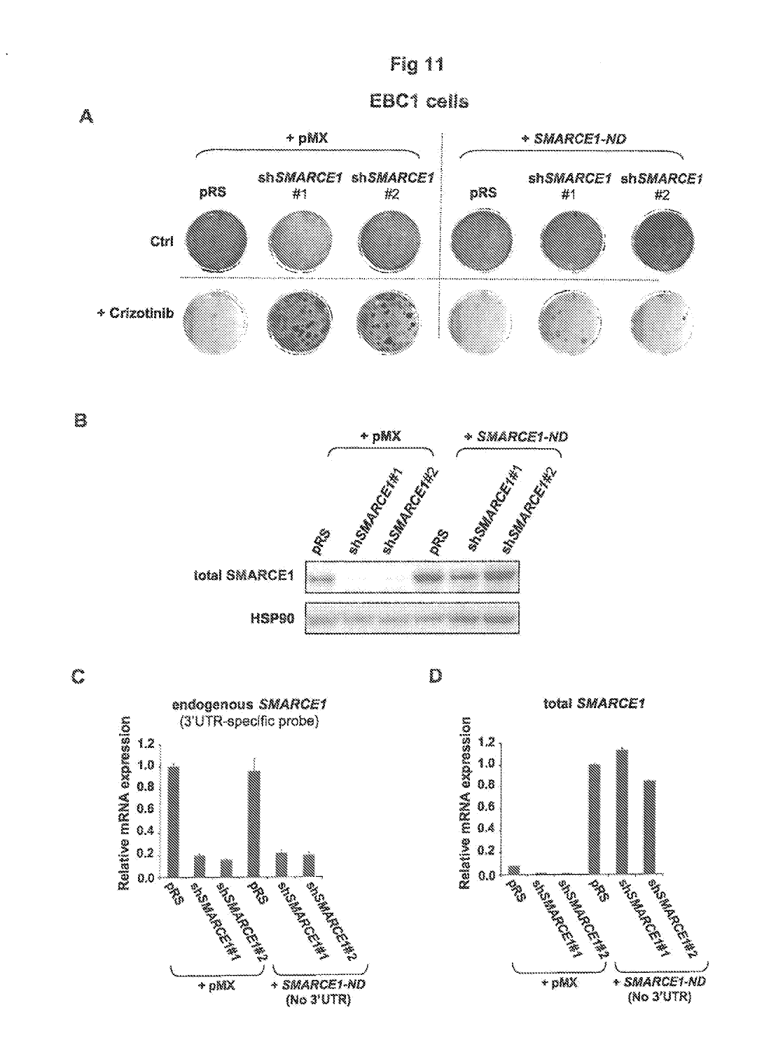

Now, Applicants addressed whether SMARCE1 is also determinant of tyrosine kinase inhibitor sensitivity in multiple NSCLC cell lines (context dependency). PC9 (EGFRdelE746-A750), H1993 (MET-amplified) and EBC-1 (MET-amplified) cells were stably infected with the retroviral shRNA constructs pRS, shSMARCE1#1 and #2 and were treated with the EGFR inhibitor geftitinib (PC9) or MET inhibitor crizotinib (H1993, EBC 1). In all cases, suppression of SMARCE1 conferred resistance to the respective inhibitors (FIGS. 9A, 10A and 11A, left panels). In parallel, the PC9, H1993 and EBC-1 cells expressing shSMARCE1#1 and #2 were infected with retroviral expression constructs encoding for the non-degradable forms of SMARCE1 (SMARCE1-ND). Reconstitution of SMARCE1 restored the sensitivity of SMARCE1-knockdown cells to the EGFR inhibitor geflitinib or MET inhibitor crizotinib (FIGS. 9A, 10A and 11A, right panels). Applicants confirmed reconstituted SMARCE1 protein levels in SMARCE1-knockdown cells by immunoblotting using an SMARCE1 specific antibody, again achieving close to endogenous level of SMARCE1 in most of the cases (FIGS. 9B, 10B and 11B). Applicants also verified a persistent knockdown of the endogenous human SMARCE1 mRNA in cells expressing the non-degradable SMARCE1 cDNAs by qRT-PCR using a human SMARCE1 3′UTR specific primer pair (FIGS. 9C, 10C and 11C). In turn, Applicants also confirmed expression of the non-degradable SMARCE1 cDNAs using an open reading frame specific primer pair detecting endogenous and ectopic (total) SMARCE1 (FIGS. 9D, 10D and 11D). It has been shown that excess SMARCE1 protein is rapidly degraded by the proteasome, suggesting that SMARCE1 protein stability requires incorporation into the SWI/SNF complex. This finding is in line with Applicants' observations from the reconstitution experiments that the protein levels of the non-degradable forms SMARCE1 were close to endogenous SMARCE1 protein level despite a significant mRNA overexpression. In conclusion, SMARCE1 is a determinant of resistance to tyrosine kinase inhibitors in multiple NSCLC cell lines.

### the Role of RAS-GAPs in the Control of Tyrosine Kinase Inhibitor Sensitivity in NSCLC Cell Lines

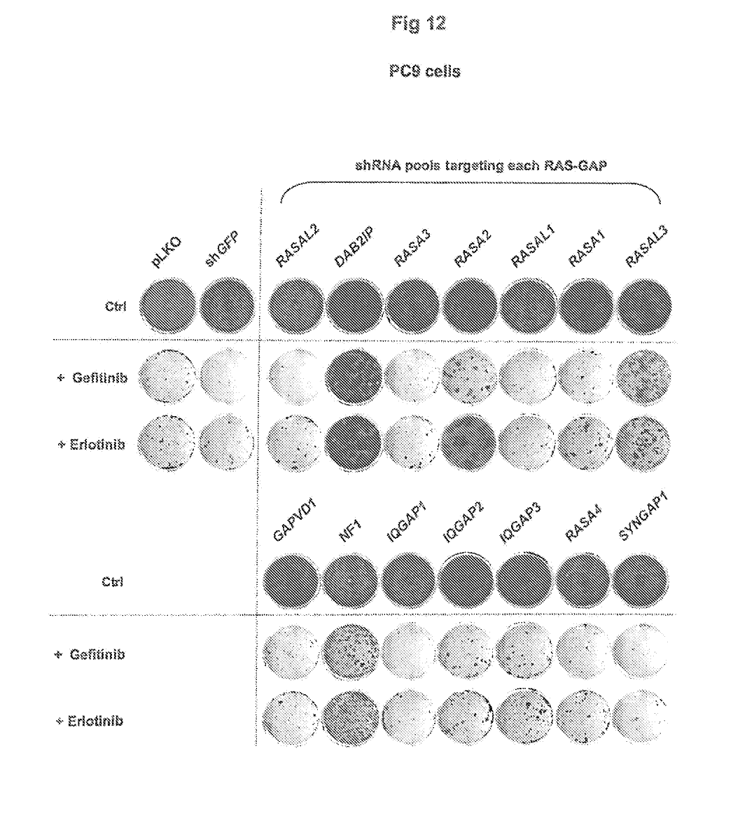

Constitutive signaling from mutated receptor tyrosine kinases such EGFR leads to activation of the RAS small GTP-binding proteins (KRAS, HRAS, NRAS). In particular KRAS is one of the most frequently mutated genes in a variety of cancers including NSCLC. RAS mutations impair the intrinsic GTPase activity and therefore prevent the conversion of active GTP-bound form into the inactive GDP-bound form (Kamoub and Weinberg, 2008). Introduction of constitutive active alleles of RAS in NSCLC cell lines renders the insensitive to tyrosine kinase inhibitors (data not shown). Therefore, inhibition of RAS is key mechanism of the efficacy of tyrosine kinase inhibitors. Applicants reasoned that direct negative regulators of RAS proteins might be critical determinants of sensitivity to tyrosine kinase inhibitors in NSCLC cell lines. The human genome encodes for 14 putative RAS-GTPase activating proteins (RAS-GAPs) that stimulate the GTPase activity of RAS proteins and promote the conversion of active GTP-loaded RAS into the inactive GDP-loaded form (Bernards, 2003). Applicants retrieved shRNAs covering the 14 putative human RAS-GAPs from the TRC shRNA collection and all shRNAs targeting the same gene were pooled together. Applicants infected PC9 cells with the 14 RAS-GAP pools in addition to the control vectors pLKO and shGFP. The cells were plated at low density and treated with the two EGFR inhibitors gefitinib and erlotinib or left untreated (FIG. 12). Several RAS-GAP pools conferred resistance to the EGFR inhibitors in the PC9 cell lines. Applicants observed the strongest resistance phenotype for the pool targeting the RAS-GAP DAB2IP. The pools directed against NF1 and RASAL3 also rendered the cells less sensitive to both EGFR inhibitors, whereas the pools targeting RASA2 exhibited inconsistent results.

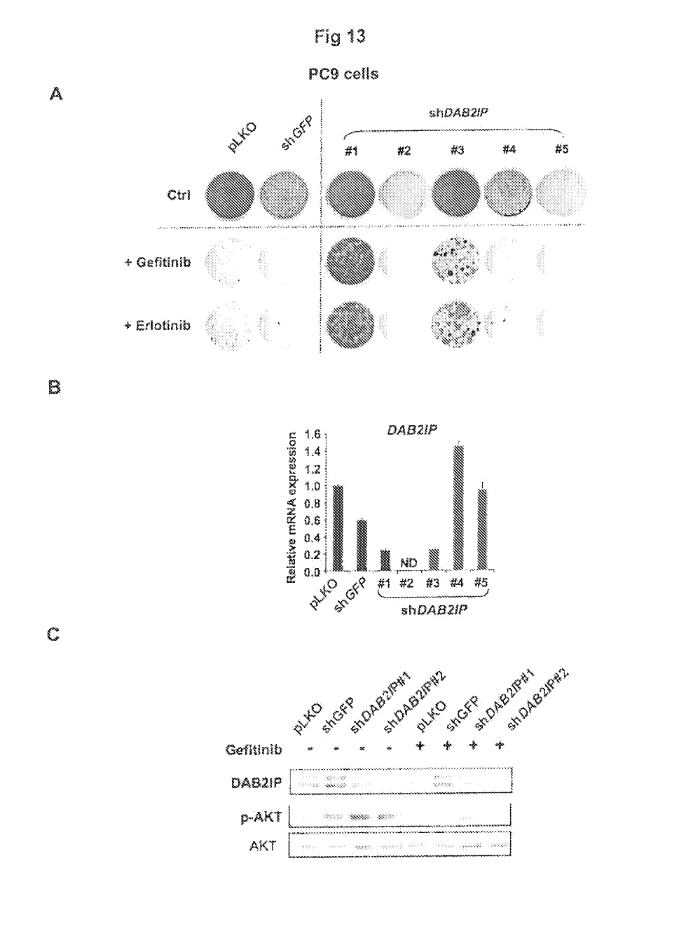

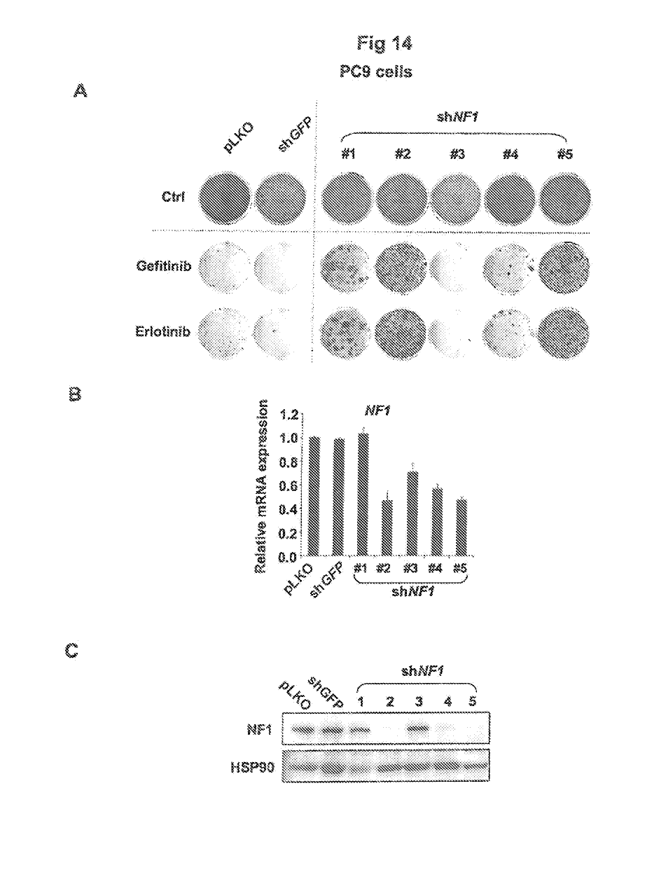

First, Applicants focused on the RAS-GAPs DAB2IP and NF1. NF1 is bona-fide tumor suppressor mutated in several cancers and also causal for the hereditable disease neurofibromatosis type I, a benign tumor syndrome with strong predisposition to several malignant cancers (Cichowski and Jacks, 2001). DAP21P plays an important role in prostate cancer and loss of its expression is associated with an aggressive metastatic disease (Min et al.). To validate the results of their focused shRNA mini-screen, Applicants individually introduced the five DAB2IP shRNAs from the TRC shRNA collection into PC9 cells (FIG. 13A). Applicants noticed that shDAB2IP#2 and to a lesser extent shDAB2IP#5 exhibited toxicity. Applicants assume that this toxicity is unrelated to the suppression of DAB2IP, as shDAB2IP#5 failed to induce a knockdown of DAB2IP. The two best shRNA vectors (shDAB2IP#1 and #3) conferred resistance to the EGFR inhibitors gefitinib and erlotinib. Suppression of DAB2IP mRNA levels was confirmed by qRT-PCR (FIG. 13B). Next, Applicants addressed whether loss of DAB2IP affects the activity of downstream signaling components of the RAS pathway, in particular the phosphorylation (activation) status of AKT. Total cell lysates were prepared from control and shDAB2IP cells (PC9) in the absence or presence of gefitinib (FIG. 13C). Applicants confirmed suppression of DAB2IP protein level in shDAB2IP expressing cells. Consistent with the inhibition of RAS by RAS-GAPs, Applicants observed elevated levels of phospho-AKT in shDAB2IP cells indicating hyperactivation of downstream components of the RAS signaling cascade. Next, Applicants individually introduced the five NF1 shRNAs from the TRC shRNA collection into PC9 cells (FIG. 14A). The two best shRNA vectors (shNF1#2 and #5) conferred resistance to the EGFR inhibitors gefitinib and erlotinib. Suppression of NF1 mRNA and protein levels was confirmed by qRT-PCR and immunoblotting (FIGS. 14B and 14C). Applicants' results show that the DAB2IP and NF1 are important determinant of sensitivity NSCLC cell to EGFR inhibitors.

### Suppression of MED12 and SMARCE1 Leads to Activation of AKT Signaling in NSCLC Cells.

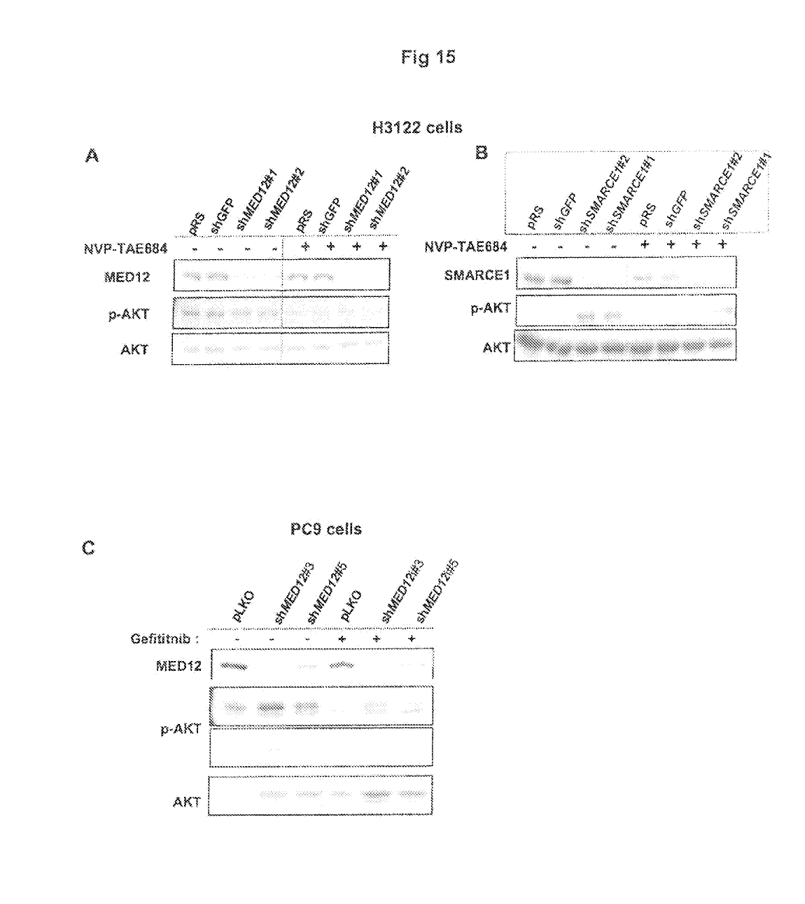

Given that loss of MED2 or SMARCE1 causes resistance to multiple tyrosine kinase inhibitors in NSCLC cell lines, Applicants asked whether the activity of downstream components of receptor tyrosine kinase signaling is altered. AKT is a key downstream component and its phosphorylation status positively correlates with its activation that can be determined by specific antibodies against the phosphorylated form of AKT. 1-13122 cells were infected with two independent controls shRNA vectors or shRNAs targeting either MED12 or SMARCE1 and confirmed loss of MED112 or SMARCE1 protein by immunoblotting (FIGS. 15A and B). The cells were also treated of left untreated with the ALK inhibitor NVP-TAE684, to address the activation status of AKT in the presence or absence of the inhibitor. Loss of SMARCE1 resulted in an increased AKT activation even in the absence of the inhibitor and consistently maintained higher levels of phosphorylated AKT in the presence of NVP-TAE684 (FIG. 15B). In conclusion, elevated activation of the key downstream component AKT upon suppression of MED12 or SMARCE1 is consistent with resistance to upstream inhibition by tyrosine kinase inhibitors. Further, Applicants could also show that loss of MED12 resulted in elevated levels of AKT phosphorylation and hence activation in PC9 cells (FIG. 15C). Applicants conclude that MED12 and SMARCE1 regulate AKT activation in multiple NSCLC lung cancer cell lines, indicating that its expression or mutation status could be an important determinant of treatment responses to tyrosine kinase inhibitors in the clinic.

### ARID1A (SMARCF) Loss Also Confers Resistance to Targeted Cancer Therapeutics in Breast Cancer.

In a related series of shRNA bar code screens in breast cancer cell lines, Applicants have asked which genes, when silenced, can contribute to inhibitors of HER2 signaling (Trastuzumab), PI-3kinase signaling, mTOR or inhibitors of both PI-3kinase/mTOR signaling.

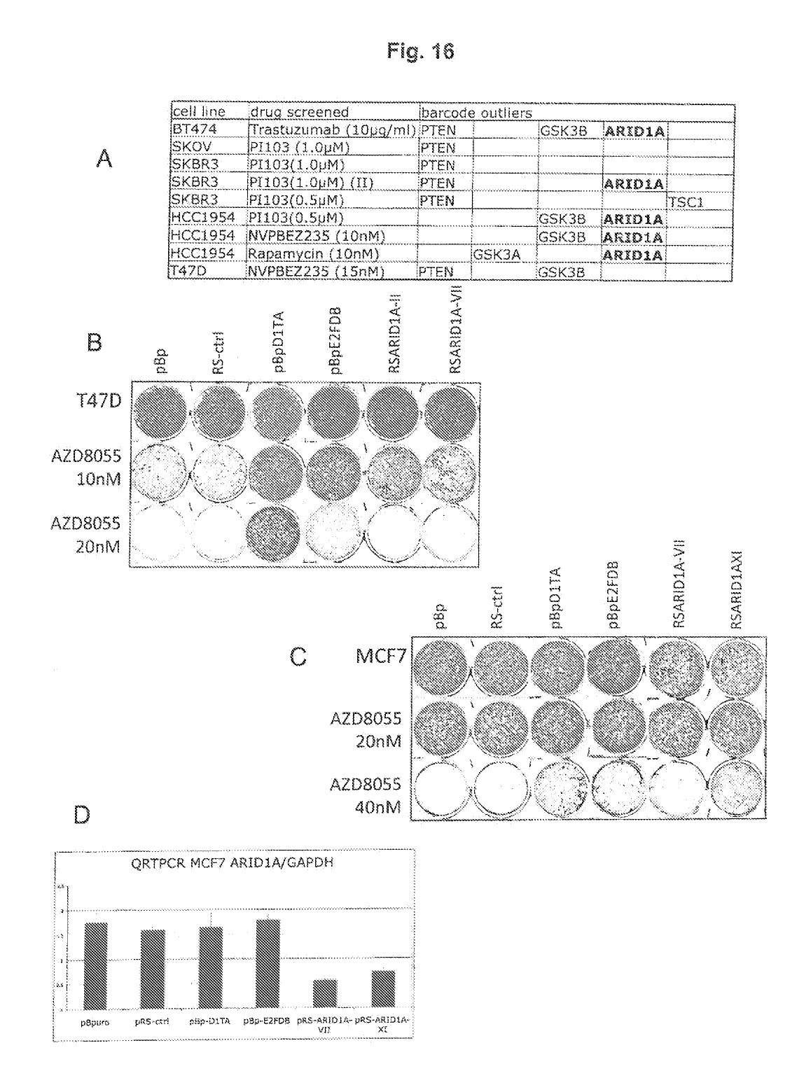

An overview of the results of these breast cancer screens is presented in FIG. 16A. Using SKBR3 breast cancer cells, Applicants found that knockdown of ARID1A (SMARCF1) conferred resistance to the dual PI-3K/mTOR inhibitors PI-103 (FIG. 16A). Similar results were obtained in HCC 1954 breast cancer cells (FIG. 16A). Moreover, knockdown of ARID1A also conferred resistance to the dual specificity PI-3kinase/mTOR inhibitors NVPBEZ235 and the mTOR inhibitor rapamycin in HCC 1954 (FIG. 16A). Moreover, when Applicants tested the ability of ARID1A knockdown vectors to confer resistance to a highly selective mTOR inhibitor drug (AZD8055) in two additional breast cancer cell lines, Applicants found that suppression of ARID1A also conferred resistance to AZD8055 in T47D and MCF7 breast cancer cells FIGS. 16B and C). That the ARID1A shRNA vectors indeed suppress the mRNA levels of the ARID1A gene is shown in FIG. 16D.

Applicants conclude that ARID1A is also a determinant of response to PI-3kinase/mTOR inhibition in breast cancer.

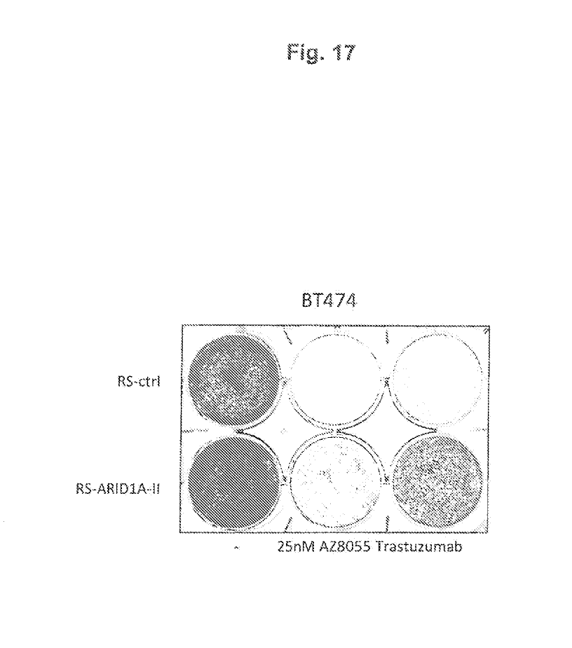

Finally, Applicants tested whether suppression of ARID1A could confer resistance to the HER2 inhibitory drug Trastuzumab (Herceptin). Applicants used the naturally HER2 amplified human breast cancer cell line BT474, which Applicants have previously shown to be highly sensitive to trastuzumab. FIG. 17 shows that knockdown of ARID1A by shRNA conferred resistance to both AZD8055 and Trastuzumab, suggesting that ARID1A is also a biomarker of response to HER2 targeted drugs like trastuzumab and lapatinib.

### REFERENCES

Bernards, A. (2003). GAPs galore! A survey of putative Ras superfamily GTPase activating proteins in man and Drosophila. Biochim Biophys Acta 1603, 47-82.
Berns, K., Hijmans, E. M., Mullenders, J., Brummelkamp, T. R., Velds, A., Heimerikx, M., Kerkhoven, R. M., Madiredjo, M., Nijkamp, W., Weigelt, B., et al. (2004). A large-scale RNAi screen in human cells identifies new components of the p53 pathway. Nature 428, 431-437.
Brummelkamp, T. R., Bernards, R., and Agami, R. (2002). A system for stable expression of short interfering RNAs in mammalian cells. Science 296, 550-553.
Brummelkamp, T. R., Fabius, A. W., Mullenders, J., Madiredjo, M., Velds, A., Kerkhoven, R. M., Bernards, R., and Beijersbergen, R. L. (2006). An shRNA barcode screen provides insight into cancer cell vulnerability to MDM2 inhibitors. Nat Chem Biol 2, 202-206.
Choi, Y. L., Soda, M., Yamashita, Y., Ueno, T., Takashima, J., Nakajima, T., Yatabe, Y., Takeuchi, K., Hamada, T., Haruta, H., et al. (2010) EML4-ALK mutations in lung cancer that confer resistance to ALK inhibitors. N Engi J Med363, 1734-1739.
Cichowski, K., and Jacks, T. (2001). NF1 tumor suppressor gene function: narrowing the GAP. Cell 104, 593-604.
Ding, N., Zhou, H., Esteve, P. O., Chin, H. G., Kim, S., Xu, X., Joseph, S. M., Friez, M. J., Schwartz, C. E., Pradhan, S., et al. (2008). Mediator links epigenetic silencing of neuronal gene expression with x-linked mental retardation. Mol Cell 31, 347-359.
Echeverri, C. J., Beachy, P. A., Baum, B., Boutros, M., Buchholz, F., Chanda, S. K., Downward, J., Ellenberg, J., Fraser, A. G., Hacohen, N., et al. (2006). Minimizing the risk of reporting false positives in large-scale RNAi screens. Nat Methods 3, 777-779.
Engelman, J. A., Zejnullahu, K., Mitsudomi, T., Song, Y., Hyland, C., Park, J. O., Lindeman, N., Gale, C. M., Zhao, X., Christensen, J., et al. (2007). MET amplification leads to gefitinib resistance in lung cancer by activating ERBB3 signaling. Science 316, 1039-1043.
Galkin, A. V., Melnick, J. S., Kim, S., IHood, T. L., Li, N., Li, L., Xia, G., Steensma, R., Chopiuk, G., Jiang, J., et al. (2007). Identification of NVP-TAE684, a potent, selective, and efficacious inhibitor of NPM-ALK. Proc Natl Acad Sci USA 104, 270-275.
Hammerman, P. S., Janne, P. A., and Johnson, B. E. (2009). Resistance to Epidermal Growth Factor Receptor Tyrosine Kinase Inhibitors in Non-Small Cell Lung Cancer. Clin Cancer Res 15, 7502-7509.
Holzel, M., Huang, S., Koster, J., Ora, I., Lakeman, A., Caron, H., Nijkamp, W., Xie, J., Callens, T., Asgharzadeh, S., et al. (2010) NF1 is a tumor suppressor in neuroblastoma that determines retinoic acid response and disease outcome. Cell 142, 218-229.
Jackman, D., Pao, W., Riely, G. J., Engelman, J. A., Kris, M. G., Janne, P. A., Lynch, T., Johnson, B. E., and Miller, V. A. (2010) Clinical definition of acquired resistance to epidermal growth factor receptor tyrosine kinase inhibitors in non-small-cell lung cancer. J Clin Oncol 28, 357-360.
Jones, S., Wang, T. L., Shih Ie, M., Mao, T. L., Nakayama, K., Roden, R., Glas, R., Slamon, D., Diaz, L. A., Jr., Vogelstein, B., et al. (2010) Frequent mutations of chromatin remodeling gene ARID1A in ovarian clear cell carcinoma. Science 330, 228-231.
Karnoub, A. E., and Weinberg, R. A. (2008). Ras oncogenes: split personalities. Nat Rev Mol Cell Biol 9, 517-531.
Kobayashi, S., Boggon, T. J., Dayaram, T., Janne, P. A., Kocher, O., Meyerson, M., Johnson, B. E., Eck, M. J., Tenen, D. G., and Halmos, B. (2005). EGFR mutation and resistance of non-small-cell lung cancer to gefitinib. N Engl J Med 352, 786-792.
Koivunen, J. P., Mermel, C., Zejnullahu, K., Murphy, C., Lifshits, E., Holmes, A. J., Choi, H. G., Kim, J., Chiang, D., Thomas, R., el al. (2008). EML4-ALK fusion gene and efficacy of an ALK kinase inhibitor in lung cancer. Clin Cancer Res 14, 4275-4283.
Kwak, E. L., Bang, Y. J., Camidge, D. R., Shaw, A. T., Solomon, B., Maki, R. G., Ou, S. H., Dezube, B. J., Janne, P. A., Costa, D. B., et al. (2010) Anaplastic lymphoma kinase inhibition in non-small-cell lung cancer. N Engl J Med 363, 1693-1703.
Lynch, T. J., Bell, D. W., Sordella, R., Gurubhagavatula, S., Okimoto, R. A., Brannigan, B. W., Harris, P. L., Haserlat, S. M., Supko, J. G., Haluska, F. G., et al. (2004). Activating mutations in the epidermal growth factor receptor underlying responsiveness of non-small-cell lung cancer to gefitinib. N Engl J Med 350, 2129-2139.
Maemondo, M., Inoue, A., Kobayashi, K., Sugawara, S., Oizumi, S., Isobe, H., Gemma, A., Harada, M., Yoshizawa, H., Kinoshita, I., et al. (2010) Gefitinib or chemotherapy for non-small-cell lung cancer with mutated EGFR. N Engl J Med 362, 2380-2388.
Malik, S., and Roeder, R. G. (2010) The metazoan Mediator co-activator complex as an integrative hub for transcriptional regulation. Nat Rev Genet 11, 761-772.
Medina, P. P., Romero, O. A., Kohno, T., Montuenga, L. M., Pio, R., Yokota, J., and Sanchez-Cespedes, M. (2008). Frequent BRG1/SMARCA4-inactivating mutations in human lung cancer cell lines. Hum Mutat 29, 617-622.
Min, J., Zaslavsky, A., Fedele, G., McLaughlin, S. K., Reczek, E. E., De Raedt, T., Guney, 1., Strochlic, D. E., Macconaill, L. E., Beroukhim, R., et al. (2010) An oncogene-tumor suppressor cascade drives metastatic prostate cancer by coordinately activating Ras and nuclear factor-kappaB. Nat Med 16, 286-294.
Pao, W., and Chmielecki, J. (2010) Rational, biologically based treatment of EGFR-mutant non-small-cell lung cancer. Nat Rev Cancer 10, 760-774.
Reisman, D., Glaros, S., and Thompson, E. A. (2009). The SWI/SNF complex and cancer. Oncogene 28, 1653-1668.
Risheg, H., Graham, J. M., Jr., Clark, R. D., Rogers, R. C., Opitz, J. M., Moeschler, J. B., Peiffer, A. P., May, M., Joseph, S. M., Jones, J. R., et al. (2007). A recurrent mutation in MED12 leading to R961W causes Opitz-Kaveggia syndrome. Nat Genet 39, 451-453.
Rodriguez-Nieto, S., Canada, A., Pros, E., Pinto, A. I., Torres-Lanzas, J., Lopez-Rios, F., Sanchez-Verde, L., Pisano, D. G., and Sanchez-Cespedes, M. (2010) Massive parallel DNA pyrosequencing analysis of the tumor suppressor BRG1/SMARCA4 in lung primary tumors. Hum Mutat.
Rosell, R., Moran, T., Queralt, C., Porta, R., Cardenal, F., Camps, C., Majem, M., Lopez-Vivanco, G., Isla, D., Provencio, M., et al. (2009). Screening for epidermal growth factor receptor mutations in lung cancer. N Engl J Med 361, 958-967.
Rudin, C. M., Avila-Tang, E., Harris, C. C., Herman, J. G., Hirsch, F. R., Pao, W., Schwartz, A. G., Vahakangas, K. H., and Samet, J. M. (2009). Lung cancer in never smokers: molecular profiles and therapeutic implications. Clin Cancer Res 15, 5646-5661.
Schwartz, C. E., Tarpey, P. S., Lubs, H. A., Verloes, A., May, M. M., Risheg, H., Friez, M. J., Futreal, P. A., Edkins, S., Teague, J., et al. (2007). The original Lujan syndrome family has a novel missense mutation (p.N1007S) in the MED12 gene. J Med Genet 44, 472-477.
Sharma, S. V., Bell, D. W., Settleman, J., and Haber, D. A. (2007). Epidermal growth factor receptor mutations in lung cancer. Nat Rev Cancer 7, 169-181.
Sharma, S. V., and Settleman, J. (2007). Oncogene addiction: setting the stage for molecularly targeted cancer therapy. Genes Dev 21, 3214-3231.
Soda, M., Choi, Y. L., Enomoto, M., Takada, S., Yamashita, Y., Ishikawa, S., Fujiwara, S., Watanabe, H., Kurashina, K., Hatanaka, H., et al. (2007). Identification of the transforming EML4-ALK fusion gene in non-small-cell lung cancer. Nature 448, 561-566.
Varela, I., Tarpey, P., Raine, K., Huang, D., Ong, C. K., Stephens, P., Davies, H., Jones, D., Lin, M. L., Teague, J., et al. Exome sequencing identifies frequent mutation of the SWI/SNF complex gene PBRMI in renal carcinoma. Nature 469, 539-542.
Versteege, I., Sevenet, N., Lange, J., Rousseau-Merck, M. F., Ambros, P., Handgretinger, R., Aurias, A., and Delattre, 0. (1998). Truncating mutations of hSNF5/INI1 in aggressive paediatric cancer. Nature 394, 203-206.

### Experimental Procedures

### shRNA Barcode Screen

The human NKI shRNA library and the barcode screen were performed as described (Berns et al., 2004; Brummelkamp et al., 2006). Additional details can be found at http://www(dot)screeninc(dot)nki(dot)nl.

### Cell Proliferation Assays

Single cell suspensions of the lung cancer cell lines were seeded into 6-well plates (2×104 cells/well) and cultured both in the absence and presence of the ALK inhibitors. At the endpoints of colony formation assays, cells were fixed with formaldehyde, stained with crystal violet (0,1% w/v) and photographed. All relevant assays were performed independently at least three times. All knockdown and overexpression experiments were done by retroviral or lentiviral infections.

### Cell Culture and Viral Transduction

H3122, PC9, HI 1993, EBC-1 and H3255 cells were cultured in RPMI with 8% heat-inactivated fetal bovine serum, penicillin and streptomycin at 5% CO2. 293T, Phoenix cells and A375 were cultured in DMEM with 8% heat-inactivated fetal bovine serum, penicillin and streptomycin at 5% CO2. Subclones of each NSCLC cell line expressing the murine ecotropic receptor were generated and used for all experiments shown. Retroviral infections were performed using Phoenix cells as producers of retroviral supernatants using 2.5-3 μg of plasmid DNA as described (http://www(dot)stanford(dot)edu/group/nolan/retroviralsystems/phx(dot)html). 293T cells were used as producers of lentiviral supernatants by co-transfecting 3rd generation lentiviral packaging constructs (2 μg of plasmid DNA) along with the pLKO shRNA vectors (2 μg of plasmid DNA). For transfections of 293T cells, Applicants seeded 1.8×10⁶ cells in a 6-well dish in the morning and transfected the cells 6-8 hours later. For transfections of Phoenix cells, Applicants seeded 1.0×10⁶ cells in a 6-well dish in the morning and transfected the cells 6-8 hours later. Cells were refreshed the next day in the morning and afternoon. Viral supernatant was harvested the day thereafter for infections of the target cells. The calcium phosphate method was used for the transfection of Phoenix and 293T cells. Infected NSCLC cells were selected for successful retroviral integration using 2 μg/ml of puromycin.

### Reagents and Antibodies

NVP-TAE648 (S1108), gefitinib (S1025), erlotinib (S1023) and crizotinib (S1068) were purchased from Selleck Chemicals. Antibodies against NF1 (SC-67) andHSP90 (H-114) were from Santa Cruz Biotechnology; antibodies against MED12 (A300-774A), SMARCE1 (A300-810A), DAB2IP (A302-439A) and NF1 (A300-140A) were from Bethyl Laboratories. Antibodies against p-AKT Ser473 (#4051) and total AKT (#9272) were from Cell Signaling. The antibody against ARID1A (H00008289-M01) was from Abnova.

### Plasmids

All retroviral shRNA vectors were generated by ligating synthetic oligonucleotides (Invitrogen) against the target genes into in the pRetroSuper (pRS) retroviral vector as described (Brummelkamp et al., 2002). The following RNAi target sequences were used for this study.

| | |
|---|---|
| shGFP | GCTGACCCTGAAGTTCATC |
| shMED121#1 | GTACCATGACTCCAATGAG |
| shMED12#2 | GGAAGAGGTGTTTGGGTAC |
| shMED12#3 | GGAGGAACTGCTTGTGCAC |
| shARID1A#1 | GGGGTGAGCTGCAACAAAG |
| shARID1A#2 | AGGAGAAGCTGATCAGTAA |
| shSMARCE1#1 | GGAGAACCGTACATGAGCA |
| shSMARCE1#2 | GGAAGAAAGTCGACAGAGA |

All lentiviral shRNA vectors (TRCN number) were retrieved from the arrayed human TRC shRNA library. Additional information about the shRNA vectors can be found at http://www(dot)broadinstitute(dot)org/rnai/public/clone/search using the TRCN number.

| | | |
|---|---|---|
| pLKO_control | | No hairpin insert |
| shGFP | | GCAAGCTGACCCTGAAGTTCA |
| shMED12_TRC#1 | TRCN0000018574 | GCAGCATTATTGCAGAGAAAT |
| shMED12_TRC#2 | TRCN0000018575 | GCTGTTCTCAAGGCTGTGTTT |
| shMED12_TRC#3 | TRCN0000018576 | CGGGTACTTCATACTTTGGAA |
| shMED12_TRC#4 | TRCNO000018577 | GCAGTTCATCTTCGACCTCAT |
| shMED12_TRC#5 | TRCN0000018578 | GCAGAGAAATTACGTTGTAAT |
| shNF1_TRC#1 | TRCN0000039713 | CCATGTTGTAATGCTGCACTT |
| shNF1_TRC#2 | TRCN0000039714 | GCCAACCTTAACCTTTCTAAT |
| shNF1_TRC#3 | TRCN0000039715 | CCTCACAACAACCAACACTTT |
| shNF1_TRC#4 | TRCN0000039716 | CCTGACACTTACAACAGTCAA |
| shNF1_TRC#5 | TRCN0000039717 | GCTGGCAGTTTCAAACGTAAT |
| shDAB2IP_TRC#1 | TRCN0000001457 | GTAATGTAACTATCTCACCTA |
| shDAB2IP_TRC#2 | TRCN0000001458 | GACTCCAAACAGAAGATCATT |
| shDAB2IP_TRC#3 | TRCN0000001459 | GAGTTCATCAAAGCGCTGTAT |
| shDAB2IP_TRC#4 | TRCN0000001460 | CTGCAAGACTATCAACTCCTA |
| shDAB2IP_TRC#5 | TRCN0000001461 | GCACATCACTAACCACTACCT |

The mouse Med12 expression constructs were generated by the following steps:

1), An linker containing first 89 bp of Med12 open reading frame (ORF) and multiple restriction sites was cloned into pcDNA3.1(+) vector by Nhel and BamHI restriction sites and was sequence verified; The oligo sequences of the top strand for the linker is CTAGCTCGAGTCGACCATGGCGGCTTTCGGGATCTTGAGCTATGAACACCGACCCCT GAAGCGGCTGCGGCTGGGGCCTCCCGATGTGTACCCTCAG and the bottom strand is GATCCTGAGGGTACACATCGGGAGGCCCCAGCCGCAGCCGCTTCAGGGGTCGGTGT TCATAGCTCAAGATCCCGAAAGCCGCCATGGTCGACTCGAG.

2), A PCR fragment of partial Med12 (from 89 to 1777 bp) was generated using a forward primer

(CAGGATCCCAAACAGAAGGAGGATGAACTGACGGCTTTGAATGTAA), a reverse primer (TGGGAGAAGACATCATGTCG) and a Med12 partial cDNA as the template (IMAGE id: 6830443); This PCR fragment was then cloned into the pcDNA3.1(+)-Med12 (first 89 bp) vector described in step 1 by BamHI and EcoRI restriction sites and was sequence verified. Note that a silence mutation (A to G) at 81 bp of Med12 ORF was introduced in the forward PCR primer to generate BamHI site in the PCR fragment.

3), An EcoRI/NotI fragment (containing from 1778 to 6573 bp of Med12 ORF) from the Med12 partial cDNA (IMAGE id: 6830443) was cloned into the pcDNA3.1(+)-Med12 (first 1777 bp) described above by EcoRI and NotI restriction sites to generate the pcDNA3.1 (+)-Mcd 12 (full-length).

4), The XhoI/NotI fragment containing the full-length Med12 ORF from pcDNA3.1(+)-Med12 was then cloned into the retroviral expression vector pMX-IRES-blasticidine using the XhoI and NotI restriction sites.

The human SMARCE1 expression construct and the non-degradable (ND) forms of were generated by PCR amplifying SMARCE1 from H3122 cDNA using the following primers:

Forward, GTACGAATTCCACCatgtcaaaaagaccatcttatgc;

Reverse, gaataagtgttgccttgttttgtgCTCGAGACTG. The fragment was cloned into the retroviral expression vector pMX-IRES-blasticidine using the EcoRI and XhoI restriction sites in the multiple cloning site and sequence verified. The SMARCE1-ND that is resistant against shSMARCE1#1 was generated by site directed mutagenesis using the following primer pair:

Forward, GCATOGAGAAAGGAGAGCCATATATGAGCATCAGCCTG;

Reverse, CAGGCTGAATGCTCATATATGGCTCTCCTTTCTCCATGC.

The SMARCE1-ND that is resistant against shSMARCE1#2 was generated by site directed mutagenesis using the following primer pair:

Forward, GAAGCTGCTTTAGAGGAGGAGAGCCGACAGAGACAATCTC;

Reverse, GAGATTGTCTCTGTCGGCTCTCCTCCTCTAAAGCAGCTTC. Both SMARCE1-ND clones were sequence verified.

Quantitative RT-PCR (qRT-PCR)

QRT-PCR assays were carried out to measure mRNA levels of genes using 7500 Fast Real-Time PCR System (Applied Biosystems). Total RNA was isolated using Trizol (Invitrogen) and 1 μg of total RNA was used for cDNA synthesis using superscrpipt 11 reverse transcriptase (Invitrogen) and random hexamer primers (Invitrogen). Relative mRNA levels of each gene shown were normalized to the expression of the house keeping gene GAPDH. The sequences of the primers for assays using SYBR® Green master mix (Roche) are listed below (h, human: m, mouse).

| | |
|---|---|
| hGAPDH_QPCR_Forward | AGGTGAAGGTCGGAGTCAA |
| hGAPDH_QPCR_Reverse | AATGAAGGGGTCATTGATGG |
| hNF1_QPCR_Forward | TGTCAGTGCATAACCTCTTGC |
| hNT1_QPCR_Reverse | AGTGCCATCACTCTTTTCTGAAG |
| hMED12_QPCR_Forward | GCTGGTGCACATAGCCACT |
| hMED12_QPCR_Reverse | TACTCCAGCCAGCCTTACCA |
| mMed12_QPCR_Forward | TCAGGCAGTGGGATTACAATGA |
| mMed12_QPCR_Reverse | TCCAGGGCGTATTTTCTCAAAAC |
| hSMARCE1_QPCR_Forward | CGGCTTATCTGGTGGCTTT |
| hSMARCE1_QPCR_Reverse | AACAACTACAGGCTGGGAGG |
| hSMARCE1_3′UTR_QPCR_Forward | GGCTTTTGGACCATTTAGCA |
| hSMARCE1_3′UTR_QPCR_Reverse | GAGGCTTTCAGCAGTTGAGG |
| hARID1A_QPCR_Forward | CCAACAAAGGAGCCACCAC |
| hARID1A_QPCR_Reverse | TCTTGCCCATCTGATCCATT |
| hDAB2IP_QPCR_Forward | AGCGAGACTCCTTCAGCCTC |
| hDAB2IP_QPCR_Reverse | GACCGCAACCACAGCTTC |

### REFERENCES

Berns, K., Hijmans, E. M., Mullenders, J., Brummelkamp, T. R., Velds, A., Heimerikx, M., Kerkhoven, R. M., Madiredjo, M., Nijkamp, W., Weigelt, B., et al. (2004). A large-scale RNAi screen in human cells identifies new components of the p53 pathway. Nature 428, 431-437.
Brummelkamp, T. R., Bernards, R., and Agami, R. (2002). A system for stable expression of short interfering RNAs in mammalian cells. Science 296, 550-553.
Brummelkamp, T. R., Fabius, A. W., Mullenders, J., Madiredjo, M., Velds, A., Kerkhoven, R. M., Bernards, R., and Beijersbergen, R. L. (2006). An shRNA barcode screen provides insight into cancer cell vulnerability to MDM2 inhibitors. Nat Chem Biol 2, 202-206.

### Example 2

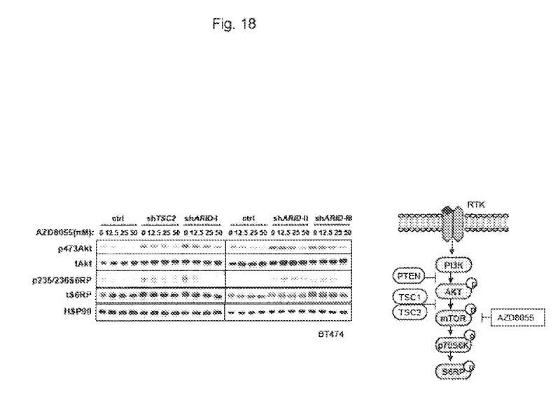

BT474 cells stably expressing either shctrl, shTSC2 or three independent shARID1A vectors were exposed to increasing amounts of the mTOR inhibitor AZD8055. After three hours, cell lysates were prepared and PI3K pathway members were immunoblotted. BT474 cells expressing the three independent shARID1A vectors maintained higher levels of phosphorylated AKT (p473-AKT) and phosphorylated S6RP (p235/236-S6RP) in the presence of increasing amounts of the mTOR inhibitor AZD8055 (FIG. 18).

Accordingly, in certain embodiments, ARID1A loss may confer resistance to PI3K/mTOR inhibitors by enhancing PI3K/mTOR pathway activation. Applicants' data suggest a link between mutation of ARID1A, commonly found in human cancer, and activation of PI3K/mTOR signaling. In other embodiments, ARID1A may serve as a biomarker for responsiveness to PI3K/mTOR targeting agents.

Having thus described in detail embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

1. A method of predicting resistance to an Akt activation and/or mTOR inhibitor in a patient in need thereof, comprising:
(a) measuring expression levels of one or more SWI/SNF complex nucleic acid and/or proteins in the patient; and
(b) comparing the expression levels of the one or more SW/SNF complex nucleic acid and/or proteins in (a) with the expression levels of one or more reference SW/SNF complex nucleic acid and/or proteins, wherein the one or more reference SW/SNF complex nucleic acid and/or proteins are from a control sample,
wherein a reduction in the expression of the one or more SW/SNF complex nucleic acid and/or proteins in comparison to the one or more reference SW/SNF complex nucleic acid and/or proteins is indicative of resistance to an Akt activation and/or mTOR inhibitor in the patient; and/or
(c) isolating nucleic acid from the patient, wherein the nucleic acid comprises one or more SWI/SNF complex DNA and/or RNA; and
(d) analyzing the nucleic acid of (c) for the presence of one or more inactivating mutations in the SWI/SNF complex DNA and/or RNA,
wherein the presence of one or more inactivating mutations in the one or more SW/SNF complex DNA and/or RNA analyzed in (d) is indicative of resistance to an Akt activation and/or mTOR inhibitor in the patient; and/or
(e) isolating protein from the patient, wherein the protein comprises one or more SW/SNF complex proteins;
(f) analyzing the activity of the one or more SW/SNF complex proteins in (e); and
(g) comparing the activity of the one or more SW/SNF complex proteins in (f) with the activity of one or more reference SW/SNF complex proteins,
wherein a difference in activity of the one or more SWI/SNF complex proteins from (f) in comparison to the one or more SW/SNF complex reference proteins in (g) is indicative of resistance to an Akt activation and/or mTOR inhibitor in the patient.

2.-19. (canceled)

20. The method of claim 1, wherein the resistance to an Akt activation and/or mTOR inhibitor is resistance to treatment with a receptor tyrosine kinase inhibitor.

21. (canceled)

22. The method of claim 1, wherein the inhibitor of Akt activation is a PI3K inhibitor.

23. The method of claim 22, wherein the PI3K inhibitor is selected from the group consisting of: NVP-BKM120, XL 147 (SAR245408), PX-866, GDC-0941, CAL-101, CNX-1351, ETP-46992, RP-5002, XL-499, and ONC-201. BEZ235, BGT226, SF1126, GSK1059615, PKI-402, PX866, GDC0941/GDC080, BKM120, NVP-BEZ235, NVP-BGT226, PF-04691502, PF-04979064, PF-05177624, PF-05197281, PF-05212384, XL147, XL765, EXEL-1229, EXEL-2400, EXEL-3751, EXEL-4251, PWT-33597, and SB2343.

24. The method of claim 1, wherein the inhibitor of mTOR is selected from the group consisting of: rapamycin/sirolimus, temsirolimus, everolimus, PP242, PP30, INK128, WYE-600, WYE-687, WYE-354, INK128, AZD8055, Torin-1, AZD2014, ridaforolimus, OSI-027, NV-128, NV-344, AP-23675, AP-23841, AP-24170, and TAFA-93. BEZ235, BGT226, SF1126, GSK1059615, PKI-402, PX866, GDC0941/GDC080, BKM120, NVP-BEZ235, NVP-BGT226, PF-04691502, PF-04979064, PF-05177624, PF-05197281, PF-05212384, XL147, XL765, EXEL-1229, EXEL-2400, EXEL-3751, EXEL-4251, PWT-33597, and SB2343.

25. The method of claim 1, wherein the resistance to an Akt activation and/or mTOR inhibitor is resistance to treatment with an inhibitor of Akt activation.

26. (canceled)

27. (canceled)

28. The method of claim 25, wherein the inhibitor of Akt activation is a receptor tyrosine kinase inhibitor.

29. The method of claim 1, wherein the SWI/SNF complex nucleic acid and/or protein is selected from the group consisting of: ARID1A, ARID1B, ARID2, SMARCA2, SMARCA4, PBRM1, SMARCC2, SMARCC1, SMARCD1, SMARCD2, SMARCD3, ACTL6A, ACTL6B, and SMARCB1.

30. The method of claim 29, wherein the SW/SNF complex nucleic acid and/or protein is ARID1A.

31.-34. (canceled)

35. The method of claim 1, wherein the patient has lung cancer.

36. The method of claim 35, wherein the lung cancer is non-small cell lung cancer.

37. The method of claim 1, wherein the patient has breast cancer.

38. The method of claim 1, wherein analyzing the nucleic acid in (d) comprises sequencing the nucleic acid or subjecting the nucleic acid to a method selected from the group consisting of: MLPA, CGH, and FISH.

39.-42. (canceled)

43. The method of claim 1, wherein the nucleic acid of (a) or (c) comprises one or more SWI/SNF complex genes.

44.-49. (canceled)

50. The method of claim 1, wherein the nucleic acid expression levels in (a) are measured using a microarray comprising a plurality of polynucleotide probes each complementary and hybridizable to a sequence in a different gene that is a SWI/SNF complex gene that is a marker for resistance to an Akt activation and/or mTOR inhibitor in a patient that has cancer.

51.-59. (canceled)

60. A kit, comprising at least one pair of primers specific for a SWI/SNF complex gene that is a marker for resistance to an Akt activation and/or mTOR inhibitor in a patient that has cancer, at least one reagent for amplification of the SWI/SNF complex gene, and instructions for use according to the method of claim 1.

61.-76. (canceled)

77. The method of claim 1, wherein the level of expression of one or more SW/SNF complex genes in (a) is measured by determination of their level of transcription, using a DNA array or quantitative RT-PCR.

78. The method of claim 1, wherein expression levels of SWI/SNF nucleic acid and/or proteins in a are measured in one or more cancer cells of the patient and wherein the nucleic acid in (c) and the protein in (e) are isolated from one or more cancers cells from the patient.

79. (canceled)

80. (canceled)

81. The method of claim 78, wherein the resistance is primary resistance to anticancer treatment.

82. The method of claim 78, wherein the resistance is secondary resistance to anticancer treatment.

83.-86. (canceled)
